(19) United States
(12) Patent Application Publication
(10) Pub. No.: US 2019/0242816 A1
(43) Pub. Date: Aug. 8, 2019

(54) APPARATUS AND METHODS FOR MEASURING FLUID ATTRIBUTES IN A RESERVOIR

(71) Applicant: EXPLORAMED NC7, INC., Mountain View, CA (US)

(72) Inventors: Arlie Conner, Portland, OR (US); Jeffery B. Alvarez, Redwood City, CA (US); Nathaniel Gaskin, Palo Alto, CA (US); Greg Kintz, Santa Cruz, CA (US); Kris Hoglund, Palo Alto, CA (US)

(21) Appl. No.: 16/372,695

(22) Filed: Apr. 2, 2019

Related U.S. Application Data
(63) Continuation of application No. PCT/US2017/049661, filed on Sep. 1, 2017.
(60) Provisional application No. 62/382,736, filed on Sep. 1, 2016.

(51) Int. Cl. G01N 21/51; G01N 33/487; G01N 33/04

(52) U.S. Cl. CPC G01N 21/51; G01F 23/292; G01N 33/04; G01N 33/487

(57) ABSTRACT

Apparatus and methods are disclosed herein for providing a sensing reservoir having one or more sensors integrated with the reservoir for measuring one or more properties of the fluid contained in the reservoir. The sensing reservoir may comprise one or more sensors configured to measure an amount of the fluid contained in the reservoir, an optical property of the fluid contained inside the reservoir, and/or a conductivity of the fluid contained inside the reservoir. The various properties or characteristics of the fluid contained in the reservoir, as determined by the one or more fluid sensors as disclosed herein, may be used to derive information about the composition or nutritional value of the fluid.

### CROSS-REFERENCE

The present application claims the benefit of U.S. Provisional Patent Application 62/382,736, filed on Sep. 1, 2016 [Attorney Docket no. 44936-716.101], the entire contents of which are incorporated herein by reference.

This application is related to the following co-pending provisional and non-provisional patent applications: U.S. patent application Ser. No. 14/221,113, filed on Mar. 20, 2014 [attorney docket no. 44936-703.201], U.S. patent application Ser. No. 14/616,557, filed on Feb. 6, 2015 [attorney docket no. 44936-704.201], U.S. patent application Ser. No. 14/793,606, filed on Jul. 7, 2015 [attorney docket no. 44936-705.201], U.S. patent application Ser. No. 14/793,613, filed on Jul. 7, 2015 [attorney docket no. 44936-706.201], U.S. patent application Ser. No. 14/858,924, filed on Sep. 18, 2015 [attorney docket no. 44936-709.201], and U.S. patent application Ser. No. 15/094,704, filed on Apr. 8, 2016 [attorney docket no. 44936-711.201], the entire contents of which are incorporated herein by reference.

### BACKGROUND OF THE INVENTION

### 1. Field of the Invention

The present invention generally relates to medical and pediatric nutrition devices and methods, and more particularly relates to devices and methods for expression and collection of human breast milk.

Breast pumps are commonly used to collect breast milk in order to allow mothers to continue breastfeeding while apart from their children. In order to understand their milk production and ensure that the production is maintained at a sufficient level, mothers often keep records of their pumping sessions manually, for example in journals or spreadsheets. Manual record keeping can be cumbersome and prone to inaccuracies or lapses in record-keeping.

It would be desirable to provide a way for mothers to automatically keep track of their milk production and the consumption of milk by their infants. It would be further desirable for the means to quantify breast milk production to be adaptable for use with various types of breast pumps. Automatic milk production quantification and inventory tracking via communication with mobile devices are further desirable for enhanced user convenience.

Further, it would be desirable to provide a way for mothers to automatically track one or more qualities of the expressed breast milk, such as its nutritional value and/or the amounts of specific substances in the milk. The content of breast milk can vary significantly from individual to individual and also for a single individual over a course of time. Understanding the composition and quality of the expressed milk can help mothers and/or their physicians make better-informed decisions regarding whether or not to feed the milk to the infants, or how a mother may work on improving the nutritional value of her milk, for example. It would be particularly desirable to provide devices and methods that enable quick, easy, and inexpensive determination of breast milk content by the user.

At least some of these objectives will be satisfied by the devices and methods disclosed below.

### SUMMARY OF THE INVENTION

The present invention generally relates to medical devices and pediatric nutrition devices and methods, and more particularly relates to devices and methods for expression and collection of human breast milk.

Apparatus and methods are disclosed herein for providing a sensing reservoir having one or more fluid sensors integrated with the reservoir for measuring one or more properties of the fluid contained in the reservoir. The sensing reservoir may comprise one or more sensors configured to measure an amount of the fluid contained in the reservoir, such as one or more capacitive sensors. Additionally or alternatively, the sensing reservoir may comprise one or more sensors configured to measure an optical property of the fluid contained inside the reservoir, such as an optical sensing unit having a light source and photodetector. Additionally or alternatively, the sensing reservoir may comprise one or more sensors configured to measure a conductivity of the fluid contained inside the reservoir, such as one or more electrodes or inductor coils. The various properties or characteristics of the fluid contained in the reservoir, as determined by the one or more fluid sensors as disclosed herein, may be used to derive information about the composition or nutritional value of the fluid.

In one aspect, an apparatus for containing and measuring a fluid comprises a reservoir configured to contain the fluid and an optical sensing unit operably coupled to the reservoir. The optical sensing unit is configured to generate measurement data indicative of one or more properties of the fluid. The optical sensing unit comprises a light source and a detector, the light source configured to emit light towards the reservoir, and the detector configured to detect an intensity of the light emanating from the reservoir.

Optionally, in any embodiment disclosed herein, the light source and the detector are arranged such that the light from the light source travels through the fluid over a path length that is less than 10 mm. The path length may be less than 5 mm. The path length may be in a range from about 1 mm to about 5 mm.

Optionally, in any embodiment disclosed herein, the light is configured to enter the reservoir at a first location of the reservoir and exit the reservoir at a second location of the reservoir positioned across the first location.

Optionally, in any embodiment disclosed herein, the light source and the detector may be arranged such that the first location is on a side wall of the reservoir, and the second location is on a bottom wall of the reservoir, such that the light travels through the fluid across a bottom corner of the reservoir. The light from the light source may be configured to pass through the first location an at oblique, downward-facing angle towards the second location. The reservoir may comprise an input light guiding structure configured to direct the light from the light source at the oblique, downward-facing angle. The reservoir may comprise an output light guiding structure configured to direct the light exiting through the second location towards the detector.

Optionally, in any embodiment disclosed herein, the reservoir may be shaped to provide a channel disposed along a bottom wall of the reservoir and protruding below the bottom wall, the channel comprising a width extending between the first location and the second location. The channel may be formed by one or more vertical channel walls coupled to a bottom channel wall. The channel may comprise a material configured to absorb at least a portion of light incident on the channel. Optionally, in any embodiment disclosed herein, the light source may be configured to emit light directly towards the first location, and wherein the detector is configured to directly receive light emanating from the second location. Optionally, in any embodiment disclosed herein, the optical sensing unit may further comprise a first lens disposed between the light source and the first location and a second lens disposed between the second location and the detector, wherein the first lens may be configured to direct light from the light source towards the first location, and the second lens may be configured to direct light from the second location towards the detector. The optical sensing unit may further comprise a first light guide disposed between the light source and the first location and a second light guide disposed between the second location and the detector, the first light guide configured to direct light from the light source towards the first location, and the second light guide configured to direct light from the second location towards the detector. The first light guide may be configured to output light in a direction that is substantially parallel to the width of the channel.

Optionally, in any embodiment disclosed herein, the sensing reservoir may further comprise one or more fluid level sensors configured to generate measurement data indicative of a level of fluid contained in the reservoir. The sensing reservoir may further comprise a processing unit operatively coupled to the one or more fluid level sensors and the optical sensing unit. The processing unit may be configured with instructions to initiate measurement with the optical sensing unit only if the level of fluid contained in the reservoir exceeds a pre-determined threshold level.

Optionally, in any embodiment disclosed herein, the optical sensing unit may be configured to measure light scattered by the fluid contained in the reservoir. The sensing reservoir may further comprise one or more fluid level sensors configured to generate measurement data indicative of a level of fluid contained in the reservoir. The sensing reservoir may further comprise a processing unit operatively coupled to the one or more fluid level sensors and the optical sensing unit, wherein the processing unit may be configured with instructions to adjust a signal measured by the detector in response to the level of fluid contained in the reservoir.

Optionally, in any embodiment disclosed herein, the apparatus may further comprise a processing unit operably coupled with the optical sensing unit, wherein the processing unit may be configured to one or more of store, process, or transmit to a remote processing unit the measurement data generated by the optical sensing unit. Optionally, in any embodiment disclosed herein, the apparatus may further comprise one or more fluid sensors configured to generate measurement data indicative of a level of fluid contained in the reservoir. The one or more fluid sensors may be operably coupled with the processing unit, and the processing unit may be configured with instructions to control measurement with the optical sensing unit in response to the level of fluid contained in the reservoir. Optionally, in any embodiment disclosed herein, the processing unit may be configured with instructions to calibrate a signal measured by the detector to generate the measurement data that is relative with respect to a calibrated value. Optionally, in any embodiment disclosed herein, the processing unit may be configured with instructions to determine one or more of a composition of the fluid, a nutritional value of the fluid, or a quality of the fluid, based on the measurement data generated by the optical sensing unit.

Optionally, in any embodiment disclosed herein, the apparatus may comprise a plurality of detectors, each of the plurality of detectors configured to receive light having a unique wavelength range, thereby enabling measurement of light absorption by the fluid at a plurality of different wavelengths. The optical sensing unit may further comprise a plurality of narrow bandpass filters disposed between the reservoir and the plurality of detectors. The optical sensing unit may comprise a plurality of light sources, each of the plurality of light sources configured to emit light having a unique wavelength range. Each of the plurality of light sources may be aligned with each of the plurality of detectors such that each pair of light source and detector forms a measurement channel for light absorption by the fluid at a unique wavelength range. The optical sensing unit may further comprise a plurality of narrow bandpass filters disposed between the plurality of light sources and the reservoir. The apparatus may further comprise a processing unit operably coupled with the optical sensing unit, wherein the processing unit may be configured with instructions to generate a discrete absorption spectrum or a continuous absorption spectrum of the fluid based on the measurement data.

Optionally, in any embodiment disclosed herein, the apparatus may comprise a pulsed driver circuit operably coupled with the light source and configured to pulse the light source during measurement with the optical sensing unit, thereby generating measurement data comprising light and dark current measurements. The apparatus may further comprise a processing unit operably coupled with the optical sensing unit, the processing unit configured with instructions to adjust a signal measured by the detector in response to dark current measurements.

In another aspect, a method of measuring a fluid contained in a reservoir comprises providing an optical sensing unit integrated with the reservoir, the optical sensing unit comprising a light source and a detector. The method further comprises emitting light from the light source, and directing the light from the light source towards a first location on the reservoir through which the light passes through to the fluid contained inside the reservoir. The method further comprises directing the light exiting the reservoir through a second location of the reservoir towards the detector. The method further comprises detecting, with the detector, an intensity of the light incident on the detector.

In another aspect, an apparatus for containing and measuring a fluid comprises a reservoir configured to contain the fluid, and one or more conductivity sensors coupled to the reservoir and configured to pass and sense a current conducted through the fluid contained in the reservoir. The apparatus further comprises circuitry operably coupled with the one or more conductivity sensors and configured to generate measurement data indicative of a conductivity of the fluid contained in the reservoir. Optionally, in any embodiment disclosed herein, the one or more conductivity sensors may comprise one or more electrodes operably coupled with the fluid contained in the reservoir. The one or more electrodes may comprise a plurality of electrodes arranged in a side-by-side, coaxial, or interdigitated configuration. The one or more electrodes may be embedded in a wall of the reservoir with the one or more electrodes in contact with the fluid contained in the reservoir. The one or more electrodes may be coated with an oxidation reagent. Optionally, in any embodiment disclosed herein, the one or more conductivity sensors may comprise one or more inductor coils operably coupled with the fluid contained in the reservoir. The one or more conductivity sensors may comprise an inductor coil disposed outside of the reservoir and adjacent to a wall of the reservoir, wherein the circuitry may comprise an LC oscillator configured to measure a change in self-resonant frequency of the LC oscillator. The one or more conductivity sensors may comprise a pair of toroidal coils coupled to wall of the reservoir and at least partially suspended in the fluid contained in the reservoir.

Optionally, in any embodiment disclosed herein, the apparatus may further comprise a computer readable memory coupled with the circuitry, the memory having stored thereon calibration data comprising conductivity measurements of a reference fluid having a known conductivity and temperature.

Optionally, in any embodiment disclosed herein, the apparatus may further comprise one or more temperature sensors coupled to the sensing reservoir and in communication with the circuitry, the one or more temperature sensors configured to measure one or more of an ambient temperature, a temperature of the fluid contained in the reservoir, or a temperature of a component of the sensing reservoir. The circuitry may be further configured to adjust the measurement data in response to one or more of the ambient temperature, the temperature of the fluid contained in the reservoir, or the temperature of a component of the sensing reservoir.

Optionally, in any embodiment disclosed herein, the apparatus may further comprise an optical sensing unit configured to measure a constituent of the fluid contained in the reservoir, wherein the circuitry may be further configured to adjust the measurement data in response to data of the constituent generated by the optical sensing unit.

In another aspect, a method of measuring a fluid contained in a reservoir comprises providing the reservoir having one or more conductivity sensors and circuitry coupled thereto integrated with the reservoir. The method further comprises driving the one or more conductivity sensors with the circuitry to pass a current through the fluid contained in the reservoir, detecting the current passed through the fluid with the one or more conductivity sensors, and generating, with the circuitry, measurement data indicative of a conductivity of the fluid contained in the reservoir.

Optionally, in any embodiment disclosed herein, the method may further comprise calibrating the one or more conductivity sensors using calibration data comprising conductivity measurements of a reference fluid having a known conductivity and temperature.

Optionally, in any embodiment disclosed herein, the method may further comprise measuring one or more of an ambient temperature, a temperature of the fluid contained in the reservoir, or a temperature of a component of the reservoir. The method may further comprise adjusting the measurement data in response to one or more of the ambient temperature, the temperature of the fluid contained in the reservoir, or the temperature of a component of the reservoir.

Optionally, in any embodiment disclosed herein, the method may further comprise measuring a constituent of the fluid contained in the reservoir, and adjusting the measurement data in response to data of the constituent.

In another aspect, an apparatus for containing and measuring a fluid comprises a reservoir configured to contain the fluid, a fluid quantity sensing unit configured to generate measurement data indicative of a quantity of the fluid contained inside the reservoir, and a fluid composition sensing unit configured to generate measurement data indicative of a composition of the fluid contained inside the reservoir. The apparatus further comprises a processing unit operably coupled to the fluid quantity sensing unit and the fluid composition sensing unit, wherein the processing unit is configured with instructions to determine a characteristic of the fluid based on both the measurement data generated by the fluid quantity sensing unit and the measurement data generated by the fluid composition sensing unit.

Optionally, in any embodiment disclosed herein, the fluid quantity sensing unit may comprise one or more capacitive sensor arrays configured to measure a level of the fluid contained inside the reservoir.

Optionally, in any embodiment disclosed herein, the fluid composition sensing unit may comprise an optical sensing unit configured to measure an absorption of light by the fluid contained inside the reservoir, wherein the processing unit may be configured with instructions to determine a composition of the fluid based on the measured absorption of light by the fluid. The processing unit may be configured with instructions to determine relative amounts of one or more of lipids, fats, triglycerides, carbohydrates, glucose, proteins, lactoferrin, organic acids, taurine, vitamins, vitamin D, minerals, sodium, zinc, copper, or iron present in the fluid, based on the measured absorption of light by the fluid. The processing unit may be configured with instructions to determine relative amounts of fats, proteins, and lactose present in the fluid, and wherein the processing unit is further configured with instructions to determine a caloric content of the fluid based on the relative amounts of fats, proteins, and lactose. The processing unit may be further configured with instructions to determine a total caloric content of the fluid contained inside the reservoir, based on the relative amounts of fats, proteins, and lactose in the fluid and the measurement data generated by the fluid quantity sensing unit.

Optionally, in any embodiment disclosed herein, the fluid composition sensing unit may comprises one or more conductivity sensors configured to measure an electrical conductivity of the fluid contained inside the reservoir, wherein the processing unit may be configured with instructions to determine a composition of the fluid based on the electrical conductivity of the fluid. The processing unit may be configured with instructions to determine a relative amount of sodium present in the fluid, based on the electrical conductivity of the fluid.

In another aspect, a method of measuring a fluid contained in a reservoir comprises providing the reservoir comprising a fluid quantity sensing unit, a fluid composition sensing unit, and a processing unit operably coupled to the fluid quantity sensing unit and the fluid composition sensing unit. The method further comprises generating, with the fluid quantity sensing unit, measurement data indicative of a quantity of the fluid contained inside the reservoir. The method further comprises generating, with the fluid composition sensing unit, measurement data indicative of a composition of the fluid contained inside the reservoir. The method further comprises determining, with the processing unit, a characteristic of the fluid based on both the measurement data generated by the fluid quantity sensing unit and the measurement data generated by the fluid composition sensing unit.

Optionally, in any embodiment disclosed herein, generating the measurement data with the fluid quantity sensing unit comprises measuring a level of the fluid contained inside the reservoir with one or more capacitive sensor arrays.

Optionally, in any embodiment disclosed herein, generating the measurement data with the fluid quantity sensing unit comprises measuring an absorption of light by the fluid contained inside the reservoir with an optical sensing unit, wherein the method further comprises determining, with the processing unit, a composition of the fluid based on the measured absorption of light by the fluid. Determining a composition of the fluid may comprise determining relative amounts of one or more of lipids, fats, triglycerides, carbohydrates, glucose, proteins, lactoferrin, organic acids, taurine, vitamins, vitamin D, minerals, sodium, zinc, copper, or iron present in the fluid, based on the measured absorption of light by the fluid. Determining a composition of the fluid may comprise determining relative amounts of fats, proteins, and lactose present in the fluid, and wherein the method further comprises determining, with the processing unit, a caloric content of the fluid based on the relative amounts of fats, proteins, and lactose. The method may further comprise determining, with the processing unit, a total caloric content of the fluid contained inside the reservoir, based on the relative amounts of fats, proteins, and lactose in the fluid and the measurement data generated by the fluid quantity sensing unit.

Optionally, in any embodiment disclosed herein, generating the measurement data with the fluid composition sensing unit comprises determining an electrical conductivity of the fluid contained inside the reservoir, wherein the method further comprises determining a composition of the fluid based on the electrical conductivity of the fluid. Determining a composition of the fluid may comprise determining a relative amount of sodium present in the fluid, based on the electrical conductivity of the fluid.

These and other embodiments are described in further detail in the following description related to the appended drawing figures.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates an exemplary embodiment of a breast pump;

FIG. 2A shows an exemplary embodiment of a sensing reservoir coupled to a pumping device;

FIG. 2B shows an exploded view of the sensing reservoir of FIG. 2A;

FIG. 2C illustrates an exemplary embodiment of the processing unit of the sensing reservoir of FIGS. 2A-2B;

FIG. 3 schematically illustrates an exemplary configuration of an optical system for measuring light scattered by a sample substance;

FIGS. 4A and 4B illustrate an exemplary embodiment of a sensing reservoir with an integrated optical sensing unit;

FIG. 5 schematically illustrates an exemplary configuration of an optical system for measuring light transmitted through a sample substance;

FIG. 6 illustrates another exemplary configuration of a sensing reservoir with an integrated optical sensing unit;

FIG. 7A shows a diagram of an exemplary driver circuit for an illumination light source for a sensing reservoir;

FIG. 7B shows a diagram of an exemplary amplifier circuit for an illumination light source for a sensing reservoir;

FIG. 8 illustrates an exemplary embodiment of a sensing reservoir comprising one or more electrodes;

FIGS. 9A-9D show exemplary configurations of electrodes suitable for incorporation with the sensing reservoir of FIG. 8;

FIGS. 10A and 10B illustrate exemplary embodiments of a sensing reservoir comprising one or more inductors;

FIG. 11 shows an exemplary method of measuring the conductivity of a fluid contained in a sensing reservoir;

FIGS. 12A-12C illustrate exemplary computing device displays suitable for incorporation with embodiments; and

FIGS. 13A-13B illustrate other exemplary displays suitable for incorporation with embodiments.

FIG. 14 illustrates another exemplary configuration of an optical sensing unit for measuring light transmitted through a sample fluid contained inside a sensing reservoir.

FIG. 15 schematically illustrates an exemplary configuration of an optical sensing unit comprising a plurality of light sources and a plurality of detectors.

FIG. 16 illustrates an exemplary embodiment of a sensing reservoir comprising an optical sensing unit as in FIGS. 14 and 15.

FIG. 17 schematically illustrates an exemplary configuration of an optical sensing unit for measuring light transmitted through a bottom corner of a reservoir.

FIG. 18A is an isometric view of an exemplary embodiment of a sensing reservoir comprising an optical sensing unit as in FIG. 17.

FIG. 18B is an exploded view of the sensing reservoir of FIG. 18A.

FIG. 18C is a detail view of section A of FIG. 18B.

FIG. 18D is a detail view of section B of FIG. 18B.

FIG. 18E is a side cross-sectional view of the sensing reservoir of FIG. 18A.

FIG. 18F is a detail view of section C of FIG. 18E.

FIG. 19A is an exploded view of another exemplary embodiment of a sensing reservoir.

FIG. 19B is a side cross-sectional view of the sensing reservoir of FIG. 19A.

FIG. 20 is a graph of regression vector data of the near-infrared absorption spectra of fat, total protein, and select lactoses.

FIG. 21 shows an exemplary method of determining a desired output value relating to a sample fluid contained in a sensing reservoir, based on data generated by the sensing reservoir.

### DETAILED DESCRIPTION OF THE INVENTION

Further details of the present disclosure are provided in the Appendix attached herewith.

Specific embodiments of the disclosed systems, devices, and methods will now be described with reference to the drawings. Nothing in this detailed description is intended to imply that any particular component, feature, or step is essential to the invention. Although the present invention primarily relates to breast milk, any description herein of expression and collection of breast milk can also be applied to other types of fluids expressed from the breast, such as colostrum, or from other glands, organs, or anatomical regions of the body. Furthermore, the disclosed embodiments may be used in other applications, particularly applications involving the measurement of any fluids collected in a collection vessel.

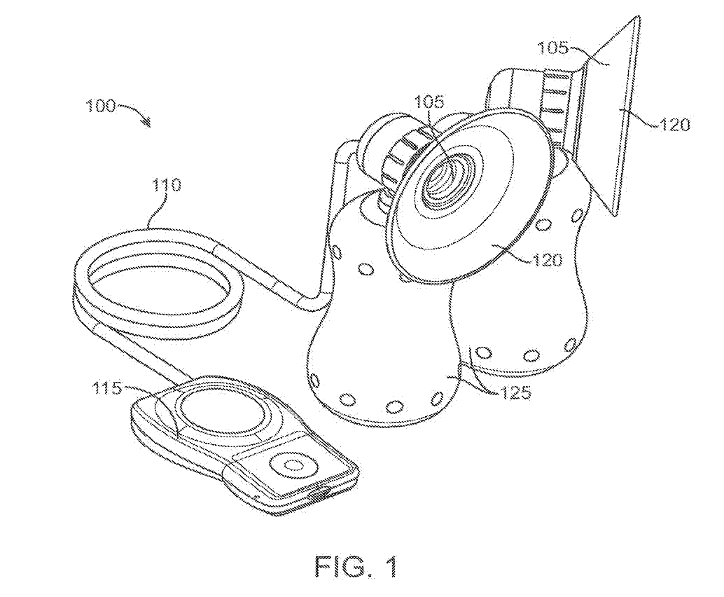

FIG. 1 illustrates an exemplary embodiment of a breast pump suitable for use with any of the present embodiments disclosed herein. Pumping device 100 (also known as an “expression apparatus”) includes one or more breast interfaces 105, a tube 110, and a controller 115 (sometimes also referred to as a “pendant unit”) operatively coupled to breast interfaces 105 through tube 110. Breast interfaces 105 include resilient and conformable flanges 120, for engaging and creating a fluid seal against the breasts, and collection vessels or reservoirs 125. Controller 115 houses the power source and drive mechanism for pumping device 100, and also contains hardware and software for various functions, such as controlling pumping device 100, milk production quantification, and communication with other devices, as described in further detail herein. Tube 110 transmits suitable energy inputs, such as mechanical energy inputs, from controller 115 over a long distance to breast interfaces 105. Breast interfaces 105 convert the energy inputs into vacuum pressure against the breasts in a highly efficient manner, resulting in the expression of milk into reservoirs 125. The device 100 may further comprise one or more sensors configured to track various characteristics of the collected fluid, as described in further detail herein. Power may be provided to the one or more sensors via a connection to the controller 115, or to another source of power.

In many instances, it can be desirable to measure and track various characteristics of milk production such as the volume or weight of the expressed milk, expression frequency (e.g., time, date), and/or expression duration. In existing approaches, the tracking of milk production is commonly accomplished by manual measurements and manual record-keeping. Sensors integrated for use with a pumping device, for example integrated with a reservoir or bottle configured to receive the pumped milk, can provide digital-based means to automatically measure and track milk production for improved convenience, efficiency, and accuracy. For example, sensors can be used to measure the volume of expressed milk as volume per unit time, or total volume per pumping session.

Sensors for producing information indicative of the quality of the expressed milk may also be provided with a pumping device, particularly integrated with the bottle or fluid reservoir configured to receive the expressed milk. For example, sensors configured to quantify the composition of the expressed milk can provide valuable information for understanding whether an infant is obtaining the appropriate amount of nutrition via the milk, whether the milk contains any undesirable contaminants, or if the mom is at risk of mastitis. This information can help mothers or clinicians identify whether additional nutrition should be supplied to the infant. Components of breast milk considered to be nutritionally important include carbohydrates such as glucose and lactose, lipids/fats such as triglycerides, proteins such as lactoferrin, organic acids such as taurine, vitamins such as vitamin D, and minerals such as sodium, zinc, copper, and iron. Analytes of particular interest may include fats, proteins, lactose, and sodium. The amount of select fat, protein, and lactose can enable a calculation of total caloric content of breast milk, which can then be used to appropriately fortify or supplement the breast milk to meet the nutritional needs of an infant. The fat content of breast milk may be particularly important for preterm infants, as they have been shown to be at high risk of receiving inadequate levels of fats from their mothers' breast milk. The whey protein content of human breast milk may also be an important factor in the development of low birth weight infants. Increased sodium concentrations in breast milk has been shown to be indicative of the occurrence of mastitis. Sensors may be provided for measuring the relative amounts of one or more of such components in the expressed milk. Sensors may also be configured to determine the estimated caloric value of the expressed milk and/or the percentage of alcohol, drugs, or other contaminants present in the milk. Such sensors may include devices that can measure the presence of certain compounds in a volume of breast milk via absorbance spectrometry and/or conductance measurements, as described in further detail herein.

One or more sensors for characterizing the quantity and/or quality of expressed milk may be coupled to the fluid collection reservoir, such as the one or more reservoirs 125 shown in FIG. 1. For example, a reservoir may comprise an integrated fluid sensing unit configured to measure one or more characteristics of the fluid contained in the reservoir. Some exemplary embodiments of a sensing reservoir are disclosed in co-pending U.S. patent application Ser. Nos. 14/616,557 and 15/094,704, the entire content of which are incorporated herein by reference.

A sensing reservoir may be supplied with its own processing unit and power source, such that the sensing capability of the reservoir may function independently of the pumping device. Providing a sensing capability in an accessory, such as a reservoir, completely separate from the pumping device can have many benefits for users. The sensing reservoir may be adaptable for use with various pumping devices, including many commercially available systems, providing a great range of flexibility for users. For example, a user may choose to add the sensing reservoir to a pumping system she already owns, in order to gain the benefits provided by an automatic fluid sensing function. In addition, in case of failure of one or more of its components, a stand-alone sensing reservoir may be easier to repair or replace than a sensor integrated into a pumping system.

The sensing reservoirs described herein may comprise collection vessels configured to couple to a pumping device for collecting expressed breast milk, such as reservoir 125 as shown in FIG. 1. Alternatively or in combination, the reservoirs may comprise bottles configured to couple to an outlet mechanism, for example a baby bottle coupled to a feeding nipple for feeding an infant. The sensing reservoirs can include one or more sensors for generating measurement data indicative of one or more characteristics of milk expression as described herein (e.g., volume of expressed milk, composition of the expressed milk, etc.). The sensors may be configured to generate the measurement data at set intervals over time, and/or at the occurrence of specific events as detected automatically or as directed by a user. Any description herein pertaining to measurement of volume can also be applied to measurements of any other characteristics, and vice-versa. Any suitable type of sensor can be used, such as accelerometers, Hall effect sensors, photodiode/LED sensors, CCD sensors, cameras and other imaging devices, capacitive sensors, strain gauges, etc., and such sensors can be used in any number and combination. The sensors can be positioned at any location on the reservoir suitable for measuring the fluid contained in the reservoir.

A processing unit may be suitably combined with any sensing reservoir described herein, wherein the processing unit may be configured to receive data from the sensor and store the data, analyze the data, and/or transmit the data to another device. A sensing reservoir having an integrated sensor and processing unit can help automate the management and monitoring of milk production, thus reducing the need for manually maintaining records related to milk production. For example, a sensing reservoir can monitor the quantity and/or quality of milk produced, and automatically process and send the data to a computing device, from which the user may easily access the information. A sensing reservoir as described herein may also be used to monitor the quantity and/or quality of milk consumed by an infant. Such a system can greatly improve convenience for the users, help reduce human errors related to manual record maintenance, and provide additional information regarding the quality of the milk that cannot easily be tracked otherwise. System and methods for managing an inventory of expressed breast milk, suitable for incorporation with the fluid measurement devices and methods disclosed in the present application, are disclosed in further detail in co-pending U.S. patent application Ser. No. 14/858,924, incorporated herein by reference in its entirety.

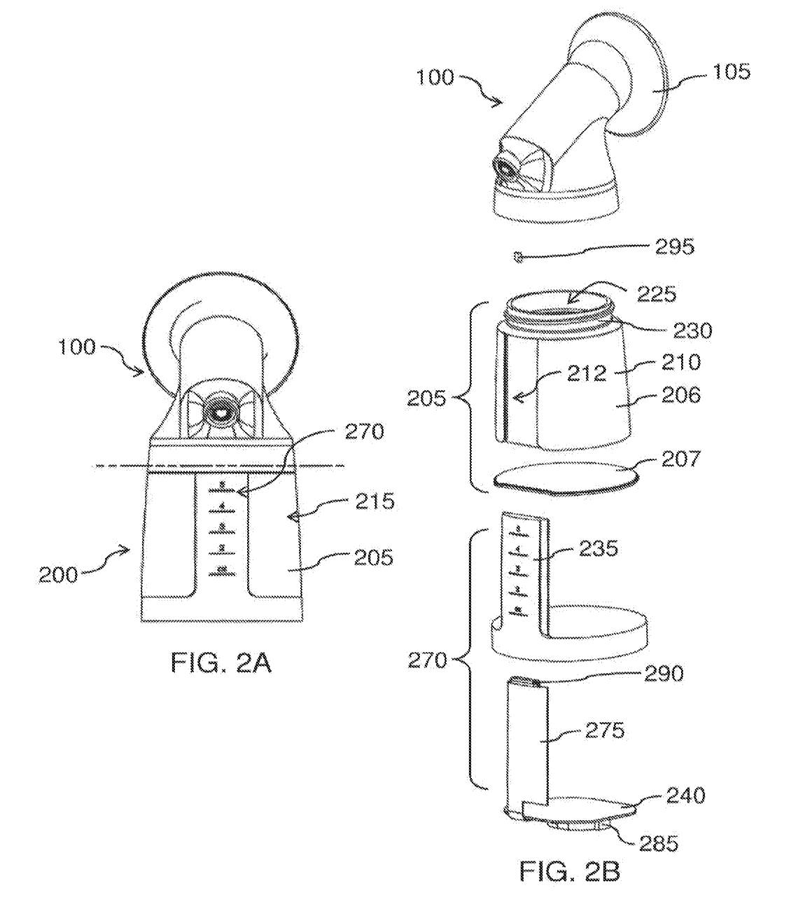

FIG. 2A shows an exemplary embodiment of a sensing reservoir 200 coupled to a pumping device 100. The sensing reservoir 200 may be used in combination with other components of a pumping device, such as the various components of pumping device 100 shown in FIG. 1. The sensing reservoir 200 comprises a reservoir 205, the reservoir having a wall 210 that defines a chamber 215. The chamber is configured to contain a fluid, such as breast milk. The chamber can have a controlled or known geometry, such that the relationship between fluid level and the volume of fluid contained is known. Such a relationship may be known for the reservoir in a substantially upright position, as shown in FIG. 2A, as well as for the reservoir in a substantially inverted position. The sensing reservoir 200 further comprises a fluid sensing unit 270, configured to generate measurement data indicative of a characteristic of the fluid contained in the reservoir. In many embodiments, the fluid sensing unit comprises one or more sensors configured to generate measurement data indicative of a volume of the fluid contained in the reservoir. Alternatively or in combination, the fluid sensing unit may comprise one or more sensors configured to generate measurement data indicative of one or more properties of the fluid, such as a composition or nutritional content of breast milk. The fluid sensing unit may be coupled to the wall of the reservoir 205, or may be disposed at any other location suitable for measuring the contained fluid.

The sensing reservoir 200 can measure the contained fluid while the sensing reservoir is in a filling state as shown in FIG. 2A, wherein the reservoir is in a generally upright position and fluid is being collected in the reservoir. For example, the sensing reservoir can operate in the filling state while the reservoir is coupled to a pumping device and collecting expressed breast milk, to measure the volume of breast milk expressed and collected in the reservoir. The sensing reservoir can further measure the fluid while the reservoir is in a draining state, wherein the reservoir is in a generally inverted position and fluid is being drained from the reservoir. For example, the sensing reservoir can operate in the draining state while the reservoir is coupled to a feeding attachment such as a feeding nipple and the contained breast milk is fed to an infant, thereby generating an indication of the volume of milk consumed by the infant. The sensing reservoir may be configured to determine the appropriate operating state, for example by sensing an orientation or tilt of the sensing reservoir, or by sensing the coupling of the reservoir to a pumping device or a feeding attachment, as described in further detail herein. Based on the determined operating state and the known geometry of the reservoir 205 and the chamber 215, appropriate algorithms may be applied in analyzing the measurement data generated by the fluid sensing unit, so as to compensate for the orientation of the reservoir, whether upright, inverted, or any other orientation, and determine the correct volume of fluid contained in the reservoir.

FIG. 2B shows an exploded view of the sensing reservoir 200 of FIG. 2A. The sensing reservoir 200 comprises the reservoir 205 and the fluid sensing unit 270 coupled to a portion of the reservoir, such as the wall 210 of the reservoir. The fluid sensing unit may comprise one or more fluid sensors 275 such as capacitive sensors, a processing unit 240 configured to receive the measurement data generated by the one or more fluid sensors, and a power source 285 configured to provide power to the processing unit and/or the one or more sensors. The fluid sensing unit may further comprise a housing 235, configured to encase the one or more fluid sensors, processing unit, and/or power source. The housing may be configured to protect the components encased therein from signal interference or damage. The housing, encasing the fluid sensors, the processing unit, and/or the power source therein, may be removably couplable to the reservoir 205, to form a reusable fluid sensing unit that can couple interchangeably to different reservoirs 205 or be removed from the reservoir while the reservoir is washed and/or sterilized.

The reservoir 205 comprises an opening 225 configured to allow passage of the fluid in and out of the reservoir. The opening 225 comprises a coupling mechanism 230, configured to removably couple to another device, such as the breast interface 105 of a pumping device or feeding nipple, via a corresponding coupling mechanism of the other device. The coupling mechanism may comprise any coupling mechanism known in the art, such as screw threads, quarter turn couplings, bayonet couplings, interference fits, and the like. In preferred embodiments, the coupling mechanism 230 comprises screw threads, so as to make the sensing reservoir widely adaptable for use with many off-the-shelf pumping devices utilizing screw threads to attach a collection vessel to the pumping device. The reservoir 205 may further comprise one or more additional openings, such as vent openings to allow passage of air and thereby facilitate passage of the fluid through the opening 225. Optionally, the reservoir 205 may comprise two or more portions that can be fixedly coupled together to collectively define the chamber 215. For example, the reservoir may comprise a body portion 206 and a bottom wall 207, wherein the bottom wall may be coupled to the body portion with a snap fit, press fit, or the like.

The fluid sensing unit 270 may comprise one or more sensors of many types and configurations. In many embodiments, the fluid sensing unit comprises one or more fluid sensors 275 configured to measure information relating to the level of fluid contained inside the reservoir 205. For example, the fluid sensing unit may comprise one or more of: a capacitive sensor configured to measure a change in capacitance affected by the fluid in proximity to the capacitive sensor; a strain gauge to measure a strain placed on the gauge by the fluid contained in the reservoir, wherein the strain gauge may be coupled to a bottom of the reservoir or to a valve disposed adjacent the opening of the reservoir; an accelerometer disposed on a valve adjacent the reservoir opening, configured to measure the motion of the valve to determine the quantity of fluid passing through; a beam-break sensor disposed adjacent the opening of the reservoir, configured to generate a signal when a fluid such as milk breaks a beam of light or other energy by passing between a beam emitter and a beam detector; an image sensor coupled to the opening of the reservoir or to a portion of the wall, and/or the image sensor configured to capture images of the fluid to quantify fluid volume.

While FIG. 2B shows a single fluid sensor 275, the fluid sensing unit may comprise a plurality of fluid sensors 275 distributed about the periphery of the reservoir in a known manner. For example, the fluid sensing unit may comprise a plurality of fluid sensors distributed about the periphery of the reservoir at a substantially equal rotational offset from one another, such that the position of the fluid in the reservoir can be accurately determined. In embodiments comprising three fluid sensors, the fluid position and reservoir orientation can be determined via triangulation of the fluid level sensed by each fluid sensor. Any of the sensors described herein may be used individually, in a plurality of one type of sensor, or in any combination of sensors.

The fluid sensing unit 270 may be coupled to the reservoir 205 in a manner that enables accurate measurement of the interior surface of the reservoir with the one or more fluid sensors 275. For example, the one or more fluid sensors 275 may be placed on the interior surface of the reservoir for direct exposure to the fluid, or the fluid sensors placed on the interior reservoir surface may be covered with a thin film coating. In preferred embodiments, the fluid sensors or sensors are embedded in the wall of the reservoir, such as in one or more recessed regions 212 of the external surface of the reservoir wall 210, to position the fluid sensors or sensors close to the interior surface of the reservoir wall. Optionally, the fluid sensor may be encased within a housing as described herein, and the recessed region may be shaped to receive a portion of the housing and the fluid sensor disposed therein, such that the external surface of the housing lays flush against the external surface of the reservoir wall. In embodiments of the fluid sensing unit comprising a plurality of fluid sensors, the reservoir may comprise a plurality of recessed regions, each of which may be shaped to receive each of the plurality of fluid sensors. The fluid sensing unit may be coupled to any suitable location on the sensing reservoir.

The processing unit 240 may be in communication with the one or more fluid sensors 275 to receive measurement data from the sensors and store the data, analyze the data, and/or transmit the data to another computing device, such as a smartphone, tablet, desktop computer, laptop computer, etc. The processing unit may perform analysis of the collected data and transmit the analyzed data to another device; alternatively, the processing unit may transmit raw measurement data to another computing device configured to perform the data analysis.

The housing 235 may comprise a material with properties such that the housing can protect the encased structures from mechanical stresses and/or water damage. In some embodiments, the housing completely encases the housed components in a leak-proof manner to protect the components from water damage. The housing may be configured to withstand mechanical stresses, extreme temperatures, and/or exposure to fluids (e.g., during milk expression or feeding or during washing of the sensing reservoir). The housing may provide electrical isolation of the fluid sensors, for example by establishing an air gap between the sensors and the housing when the sensors are encased within the housing. The housing may be coupled to the reservoir 205 fixedly (e.g., epoxy or cyanoacrylate adhesive bonding, ultrasonic welding, etc.) or removably via a releasable coupling mechanism to the reservoir.

Optionally, the sensing reservoir 200 may further comprise one or more reservoir sensors, configured to measure a position, orientation, and/or motion of the reservoir. For example, reservoir sensors may comprise one or more of accelerometers configured to detect motion of the sensing reservoir or gyroscopes configured to detect an orientation of the sensing reservoir. The one or more reservoir sensors can improve the accuracy of fluid measurement by the fluid sensing unit. Reservoir sensors that provide the orientation of the sensing reservoir can enable an algorithmic compensation for the reservoir orientation, thereby increasing the accuracy of fluid volume calculation based on the fluid levels detected by the fluid sensors. Often, the top portion of a reservoir chamber can have a different geometry than the bottom wall of the reservoir chamber, such that the translation between fluid level and contained fluid volume depends on whether the reservoir is substantially upright or inverted. Reservoir sensors configured to determine whether the reservoir is in an upright or inverted configuration can thus facilitate the selection of the correct translation algorithm in performing analysis of the fluid sensor data. Further, reservoir sensors can enable the sensing reservoir to switch from one operating state to another. For example, reservoir sensors configured to measure a position or motion of the sensing reservoir can determine when the reservoir is in an inactive/standby or “sleep” state, filling state, draining state, or in transition between one operating state to another. The fluid sensing unit can be configured to pause data collection during times the reservoir is determined to be in a standby or sleep state, so as to reduce power consumption and the collection of redundant data points. Further, the fluid sensing unit may be configured to collect data only during times the reservoir is determined to be in a stable filling state or draining state without excessive detected motion, so as to reduce the collection of unusable (e.g., excessively noisy) data points. The reservoir sensors may be disposed on any portion of the sensing reservoir. For example, the reservoir sensors may be integrated with the fluid sensing unit 270. In preferred embodiments, the reservoir sensors are disposed on the processing unit 240, and in communication with a microcontroller or microprocessor of the processing unit.

Optionally, the sensing reservoir 200 may further comprise a means for detecting the coupling of the sensing reservoir to another component, such as a pumping device, a feeding attachment, or a storage cap. The detection of the coupling may be used as a cue for the fluid sensing unit and/or the reservoir sensors to initialize the system, determine the appropriate operating state, and begin sensor interrogation, enabling the sensing reservoir to switch quickly and accurately between different operating states (e.g., standby/sleep, filling, draining) and thereby optimize the efficiency of power consumption by the sensing reservoir. For example, as shown in FIG. 2B, the means for detecting the coupling may comprise one or more proximity sensors 290 coupled to the sensing reservoir, and one or more corresponding proximity triggers 295 coupled to a component to be coupled to the reservoir (such as pumping device 100). The proximity sensors may be located near the portion of the reservoir configured to couple to the component, and the proximity triggers may be located near the portion of the portion of the component configured to couple to the sensing reservoir, such that the sensors and the triggers are brought into proximity when the sensing reservoir is coupled to the component. The proximity sensor may be configured to detect the proximity trigger when the proximity trigger is placed within a predetermined distance from the proximity sensor. When the component comprising the proximity trigger is coupled to the sensing reservoir, the proximity trigger is brought within the predetermined distance from the proximity sensor, thus enabling the proximity sensor to detect the coupling of the component to the sensing reservoir. The sensing reservoir may comprise one or more of the following proximity trigger/sensor combinations: reflective markers as triggers and light source/photodiode assembly as sensors; magnets as triggers and Hall effect sensors as sensors; magnets as triggers and reed switches as sensors. Other sensors known in the art may also be used as the proximity sensors.

The proximity sensors and proximity triggers may be provided in various configurations in order to enable identification of the component that is coupled to the sensing reservoir. Thus, the sensing reservoir may be able to distinguish between the coupling of the reservoir to a feeding attachment or to a pumping device, enabling the system to determine the operating state of the sensing reservoir (e.g., whether the reservoir is about to begin filling (when attached to pumping device) or draining (when attached to feeding attachment)). In this case, the detection of a coupling event can not only direct the system to begin interrogation of the fluid sensing unit, but also help the processing unit select the appropriate analysis algorithm for the calculation of fluid levels based on the measurement data produced by the fluid sensing unit. The proximity sensor-derived operating state information can be cross-checked against the operating state information derived from the reservoir sensors and/or the fluid sensing unit to verify the current operating state (e.g., standby, filling, draining) of the sensing reservoir. Optionally, the components to be attached to the sensing reservoir may comprise unique identifiers that are recognizable by the processing unit, such that the system may be able to detect the coupling of the reservoir to unauthorized parts and notify the user accordingly.

FIG. 2C illustrates an exemplary embodiment of the processing unit 240 of the sensing reservoir 200 of FIGS. 2A-2B. The processing unit may comprise one or more of a printed circuit board (PCB) 242 housing one or more of a microcontroller or microprocessor 244, a communication module 246, a fluid sensor connection 248, a power connection 249, a proximity sensor connection 247, and a timer 243. The processing unit may further comprise a memory (not shown). Power may be supplied to the processing unit via a power source comprising a battery, such as power source 285 shown in FIG. 2B, or a direct contact connection such as a cable or pad connectors. Alternatively or in combination, power may be supplied via an inductive charging system comprising a battery (such as power source 285 shown in FIG. 2B) and a wireless charger, which may be charged using an inductive charging method as known in the art.

The processing unit may receive signals from the fluid sensing unit through the fluid sensor connection 248, and the signals may be transmitted to the microprocessor 244. One or more reservoir sensors 241, configured to measure a position, orientation, and/or motion of the sensing reservoir as described herein, may also be disposed on the processing unit, and may transmit measured signals directly to the microprocessor 244. Optionally, the processing unit may also receive signals from one or more proximity sensors through the proximity sensor connection 247, and the signals may be transmitted to the microprocessor 244. The microprocessor may comprise a non-transitory computer readable medium comprising instructions to collect and process the signals received from the fluid sensing unit, the reservoir sensors, and/or the proximity sensors. The microprocessor may further comprise instructions to transmit the collected and/or processed signals to a memory for storage, or to the communication module 246 for transmission to another computing device. The communication module may comprise a wireless transmitter/receiver such as a Blue Tooth or a WiFi module, for example. The communication module may be configured to transmit the measurement data to another computing device, such as a mobile phone, tablet, or personal computer, for data analysis and/or display of the analyzed data to a user. Alternatively or in combination, the communication module may be configured to transmit the measurement data to a server for data analysis, and the server may transmit the analyzed data to a personal computing device for display to the user. The user may view and track the analyzed measurement data from the computing device, for example via a mobile application on a mobile phone.

In any of the embodiments, the fluid sensing unit 270 further may comprise one or more sensors in addition to fluid sensors 275 shown and described with reference to FIGS. 2A and 2B, configured to measure information relating to the quality of the fluid contained inside the reservoir 205. For example, the fluid sensing unit may comprise sensors configured to measure various physical, chemical, electrical, or optical characteristics of the fluid. In many embodiments, the fluid sensing unit may further comprise one or more photometers configured to measure the intensity of light scattered by, reflected by, or transmitted through the fluid contained inside the reservoir. Alternatively or additionally, in many embodiments, the fluid sensing unit may comprise one or more sensors configured to measure an electrical conductivity of the fluid contained inside the reservoir. The optical or electrical interrogation advantageously enables noninvasive and nondestructive sampling of the fluid contained inside the reservoir.

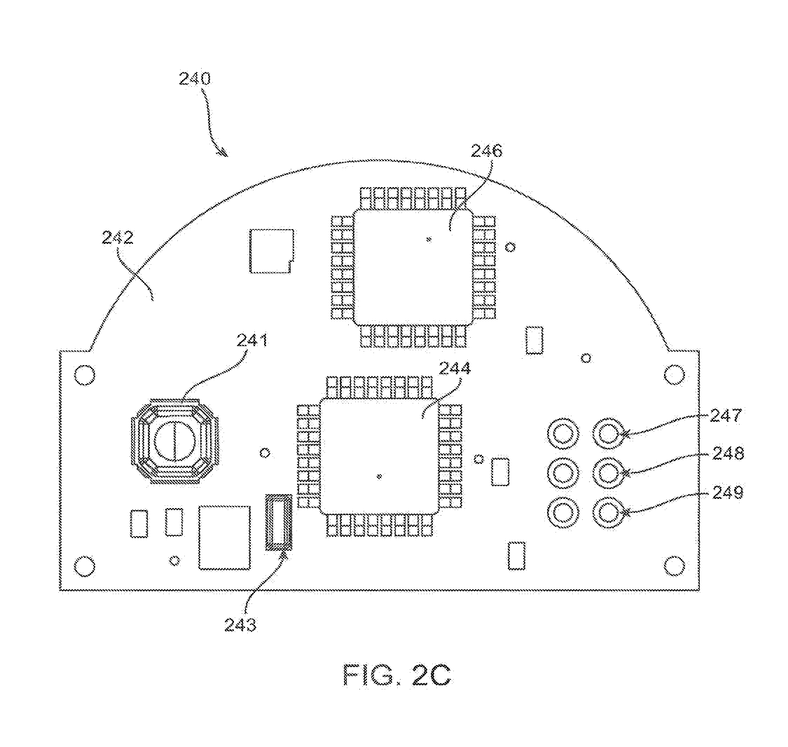

FIG. 3 schematically illustrates an exemplary configuration of an optical system 300 for measuring light scattered by a sample substance, which may be applied to any of the sensing reservoirs as described herein. The optical system 300 comprises an illumination light source 302, a first lens 304, a second lens 306, and a detector 308. The light source, which may comprise a laser, light-emitting diode (LED), lamp, any other suitable light source known in the art, or any combination thereof, may be configured to emit an illumination light beam 312 generally in the direction of the sample substance. The light source may optionally comprise a plurality of light sources, such as a plurality of LEDs having a different central emission wavelengths. The first lens 304 may be disposed between the light source 302 and the sample substance S and configured to collimate, focus, or direct the illumination light 312 towards the sample substance S. When light enters the sample, photons may collide with molecules in the sample substance, thereby causing scattering of the light. The extent of light scatter may be correlated with one or more properties of the sample substance. For example, larger and denser the molecules in the sample substance may result in a greater extent of light scattering. The scattered light 314 may be collected by the second lens 306 disposed between the sample substance and the detector 308 not in the direct path of the illumination light beam. The second lens can be configured to collimate, focus, or direct the scattered light 314 towards the detector 308. The detector, which may comprise any photodetector known in the art, can be configured to convert the amount of scattered light contacting the detector into voltages, currents, or resistance, for example.

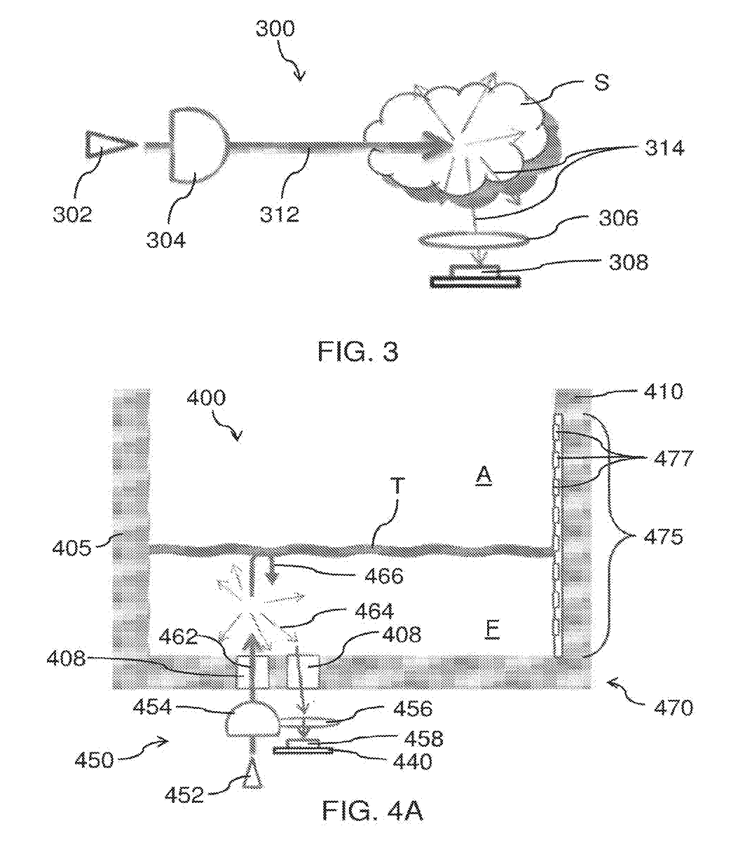

FIGS. 4A and 4B illustrate an exemplary configuration of a sensing reservoir 400 with an integrated optical sensing unit 450 for measuring light scattered by the fluid contained inside the sensing reservoir, which may apply to any of the sensing reservoirs described herein. FIG. 4A shows the sensing reservoir 400 containing fluid, such as expressed breast milk, of a relatively low volume. FIG. 4B shows the sensing reservoir 400 containing fluid of a relatively high volume. Sensing reservoir 400 may be used in combination with other components of a pumping device, such as the various components of pumping device 100 shown in FIG. 1. The sensing reservoir 400 may be similar in one or more aspects to the sensing reservoir 200 shown in FIGS. 2A-2B, and may comprise one or more features or elements described in reference to the sensing reservoir 200. For example, the sensing reservoir 400 may comprise a reservoir 405 and a fluid sensing unit 470 comprising at least one fluid sensor 475, wherein the reservoir 405, fluid sensing unit 470, and fluid sensor 475 may be similar in many aspects to reservoir 205, fluid sensing unit 270, and fluid sensor 275, respectively, shown and described with reference to FIGS. 2A and 2B. The fluid sensor 475 may comprise a capacitive sensor configured to measure a level of fluid in the reservoir by detecting a change in capacitance affected by the dielectric permittivity of the fluid in proximity to the sensor. As shown, the fluid sensor 475 may comprise an array of a plurality of capacitive sensing regions 477 extending along the height of the reservoir. The fluid sensing unit 470 may be encased within a housing and embedded within the wall 410 of the reservoir, for example within a recessed region of the external surface of the wall as described herein.

In addition to the fluid sensor 475 configured to measure a level of fluid F contained inside the reservoir 405, the fluid sensing unit 470 may further comprise an optical sensing unit 450 configured to measure light scatter by the fluid. While FIGS. 4A and 4B show the optical sensing unit 450 coupled to the bottom 407 of the reservoir 405, the optical sensing unit may be positioned at any other location of the reservoir suitable for optically interrogating the fluid F inside the reservoir. The optical sensing unit 450 may be configured to function substantially similarly to the optical system 300 shown and described with reference to FIG. 3. The optical sensing unit 450 may comprise an illumination light source 452, first lens 454, second lens 456, and detector 458. The illumination light source may comprise a single light source or a plurality of light sources. The illumination light source 452 may emit an illumination light beam 462 that is generally directed towards the fluid F inside the reservoir 405. The first lens 454, disposed between the light source 452 and the reservoir bottom 407, may collimate, focus, or otherwise direct the illumination light towards the fluid F. Light 462 entering the fluid F may be scattered by the molecules in the fluid, such that at least some of the scattered light 464 may be collected by the second lens 456, disposed between the reservoir bottom 407 and the detector 458. The second lens 456 may be configured to collimate, focus, or otherwise direct light towards the photodetector 458. The photodetector may be configured to detect an intensity of the scattered light incident on the detector, converting the incident light into voltage, current, or resistance measurements. To allow the illumination light beam 462 and the scattered light 464 to pass through the reservoir 405, the reservoir 405 may comprise an optically clear material, or optically clear windows 408 extending through the thickness of the reservoir bottom 407 and disposed over locations at which light is configured to enter and leave the reservoir.

The optical sensing unit 450 may be operably coupled to a processing unit 440, which may be similar in many aspects to processing unit 240 as described in reference to FIGS. 2B and 2C. The processing unit may be configured to receive measurement data from the photodetector 458 of the optical sensing unit, and store, analyze, and/or transmit the measurement data. The optical sensing unit 450 may further be operably coupled to a power source such as power source 285 shown in FIG. 2B. Optionally, the optical sensing unit may be encased within a protective housing such as housing 235 shown in FIG. 2B, so as to protect the optical sensing unit from physical damage and/or interference (e.g., stray light). Specifically, the optical sensing unit may be encased within a bottom wall of the housing configured to be disposed over the bottom wall of the reservoir. The housing may comprise optically clear windows disposed over locations at which light is configured to enter and leave the reservoir.

Measurements of scattered light by the optical sensing unit 450 as shown in FIGS. 4A and 4B may yield relatively low signal strength, since the portion of the illumination light beam that is scattered towards the direction of the second lens may be relatively small. In addition, a significant portion of the illumination light beam may be reflected off the top surface T of the fluid F due to the refractive index change of the top surface T to the air A above the fluid. At least some of this reflected light 466 may be collected by the second lens 456 to reach the detector 458, such that the light incident on the photodetector comprises both reflected light 466 and scattered light 464. When the fluid level is relatively low as shown in FIG. 4A, the reflected light 466 may comprise a larger portion of the total light incident on the detector than the scattered light 464. When the fluid level is relatively high as shown in FIG. 4B, the reflected light 466 may comprise a smaller portion of the total light incident on the detector than the scattered light 464. Thus, the light incident on the detector can comprise a different ratio of scattered versus reflected light depending on the level of fluid contained inside the reservoir. Accordingly, in order to correctly interpret the measurements made by the detector, it may be necessary to adjust the measured signal based on the fluid level. In a sensing reservoir comprising fluid sensors configured to measure the amount of fluid present in the reservoir, such as the sensing reservoir 400 as shown in FIGS. 4A and 4B, the fluid level determined using the fluid sensors 475 can be used to establish the path length of the light from the light source to the detector. The established path length can be used to adjust the signal obtained by the photodetector by an amount related the amount of fluid in the reservoir.

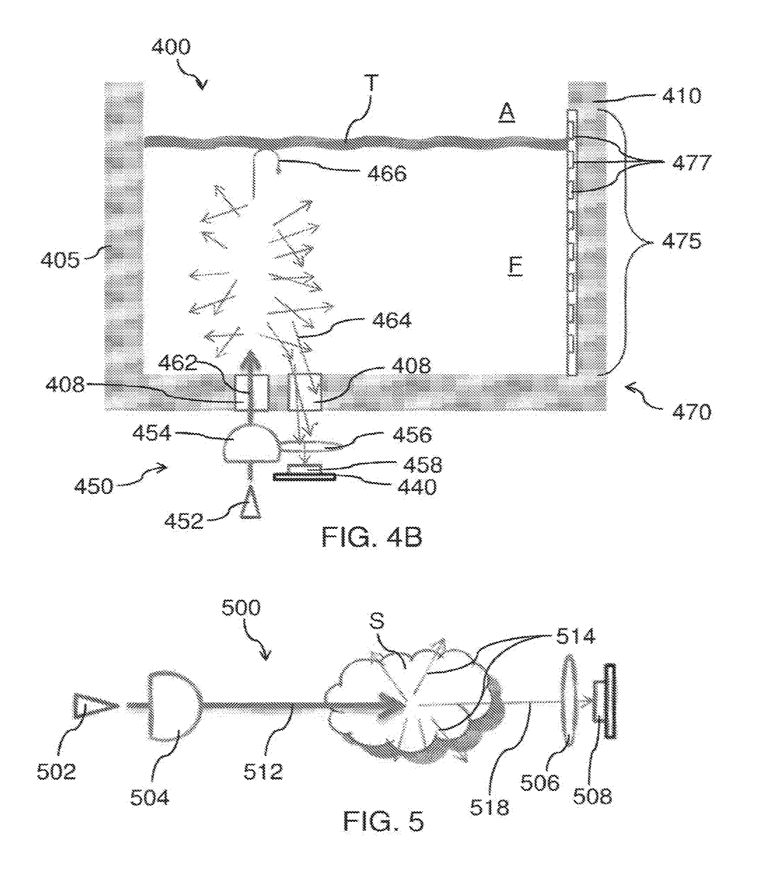

FIG. 5 schematically illustrates an exemplary configuration of an optical system 500 for measuring light transmitted through a sample substance, which may apply to any of the embodiments disclosed herein. The optical system 500 comprises an illumination light source 502, a first lens 504, a second lens 506, and a detector 508, which may be similar in many aspects to the correspondingly-named components of optical system 300 of FIG. 3. For example, the light source may optionally comprise a plurality of light sources, such as a plurality of LEDs having a different central emission wavelengths. However, in optical system 500, the second lens 506 is disposed between the sample substance S and the detector 508 substantially in the direct path of the illumination light beam 512 passing through the sample substance, such that the majority of the light focused onto the detector comprises light 518 transmitted through the sample. Compared to measuring scattered light 514, measuring transmitted light 518 can result in the detection of a stronger signal subject to less variance.

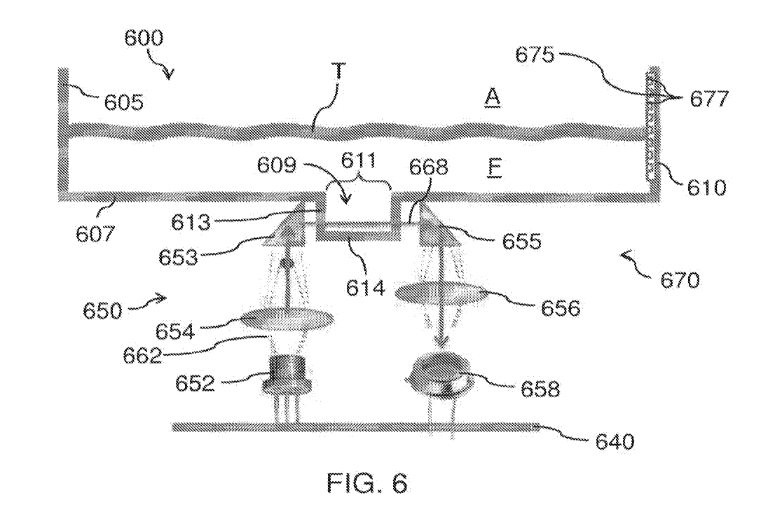

FIG. 6 illustrates an exemplary configuration of a sensing reservoir 600 with an integrated optical sensing unit 650 for measuring light transmitted through the fluid contained inside the sensing reservoir, which may apply to any of the embodiments disclosed herein. Sensing reservoir 600 may be used in combination with other components of a pumping device, such as the various components of pumping device 100 shown in FIG. 1. The sensing reservoir 600 may be similar in one or more aspects to the sensing reservoir 200 shown in FIGS. 2A-2B, and may comprise one or more features or elements described in reference to the sensing reservoir 200. For example, the sensing reservoir 600 may comprise a reservoir 605 and a fluid sensing unit 670 comprising at least one fluid sensor 675, wherein the reservoir 605, fluid sensing unit 670, and fluid sensor 675 may be similar in many aspects to reservoir 205, fluid sensing unit 270, and fluid sensor 275, respectively, shown and described with reference to FIGS. 2A and 2B. The fluid sensor 675 may comprise a capacitive sensor configured to measure a level of fluid in the reservoir by detecting a change in capacitance affected by the dielectric permittivity of the fluid in proximity to the sensor. As shown, the fluid sensor 675 may comprise an array of a plurality of capacitive sensing regions 677 extending along the height of the reservoir. The fluid sensing unit 670 may be encased within a housing and embedded within the wall 610 of the reservoir, for example within a recessed region of the external surface of the wall as described herein.

In addition to the fluid sensor 675 configured to measure a level of fluid F contained inside the reservoir 605, the fluid sensing unit 670 may further comprise an optical sensing unit 650 configured to measure light transmitted through the fluid. While FIG. 6 shows the optical sensing unit 650 coupled to the bottom 607 of the reservoir 605, the optical sensing unit may be positioned at any other location of the reservoir suitable for optically interrogating the fluid F inside the reservoir. For example, the optical sensing unit may be coupled to a side wall 610 adjacent the bottom of the reservoir, or at adjacent the opening of the reservoir 605 through which fluid enters the reservoir. The optical sensing unit 650 may be configured to function substantially similarly to the optical system 500 shown and described with reference to FIG. 5. The optical sensing unit 650 may comprise one or more illumination light sources 652, first lens 654, second lens 656, and detector 658, which may be similar in many aspects to the correspondingly-named components described in reference to FIG. 5. Further, the optical sensing unit 650 may comprise a first light guide 653 disposed between the first lens 654 and the reservoir 605, and a second light guide 655 disposed between the reservoir 605 and the second lens 656. The light guides may comprise, for example, prisms, optical fibers, or other optical components known in the art that can redirect incoming light at an off angle.

To enable the measurement of light transmitted through the fluid at a fixed, known path length, the reservoir 605 may be shaped to provide a channel 609 at the bottom 607 of the reservoir, through which the illumination light beam may be passed. The channel may be formed by one or more vertical channel walls 613 protruding downwards below the reservoir bottom 607, and coupled to a bottom channel wall 614 whose plane is disposed below the plane of the reservoir bottom 607. When fluid F begins to fill the reservoir with the reservoir in substantially vertical or upright orientation, channel 609 may become filled with the fluid. The channel 609 may have a known width 611, which sets the path length of the light through the fluid F. To allow the illumination light beam 662 and the transmitted light 668 to pass through the reservoir 605, the channel 609 may comprise an optically clear material, or optically clear windows extending through the thickness of the channel walls and disposed over locations at which light is configured to enter and leave the reservoir. Alternatively or in combination, the channel 609 may comprise a material having some moderate absorption of incident light, in order to improve the discrimination of the measurement by helping to reduce stray light. For example, the channel 609 may be constructed out of a material such as polyphenylsulfone (PP SU).

The components of the optical system 650 may be mounted vertically on a substrate of a processing unit 640, such as a PCB board. The processing unit 640 may be similar in many aspects to processing unit 240 as described in reference to FIGS. 2B and 2C. The processing unit may be configured to receive measurement data from the photodetector 658 of the optical sensing unit, and store, analyze, and/or transmit the measurement data. The light source 662 may also be coupled to the substrate of processing unit 640, wherein the processing unit may comprise circuitry configured to drive the light source, as described in further detail herein.

The optical sensing unit 450 may further be operably coupled to a power source such as power source 285 shown in FIG. 2B. Optionally, the optical sensing unit may be encased within a protective housing such as housing 235 shown in FIG. 2B, so as to protect the optical sensing unit from physical damage and/or interference (e.g., stray light). Specifically, the optical sensing unit may be encased within a bottom wall of the housing configured to be disposed over the bottom wall of the reservoir. The housing may comprise optically clear windows disposed over locations at which light is configured to enter and leave the reservoir.

In use, the illumination light source 652, which may comprise a laser, light-emitting diode (LED), lamp, any other suitable light source known in the art, may emit an illumination light beam 662 generally directed upwards towards the reservoir bottom 607. The first lens 654, disposed between the light source 652 and the first light guide 653, may collimate, focus, or otherwise direct the illumination light towards the first light guide 653. Light 662 entering the first light guide 653 may be redirected at an off angle, such that the output light from the first light guide enters the channel 609 of the reservoir 605 in a direction that is substantially parallel to the width 611 of the channel. For example, in embodiments wherein the light source and the first lens are arranged to direct the illumination light beam in a direction substantially orthogonal to the plane of the reservoir bottom 607, the first light guide may be configured to redirect the input light at about a 90° angle. Such a configuration can help ensure that the path length of the light remains constant and substantially equal to the known width 611 of the channel. The illumination light 662 exiting the first light guide 653 subsequently enters the channel 609 and travels across the width 611 of the channel, through the fluid F disposed within the channel. The illumination light therefore enters the reservoir at a first location that is on one side of the channel, and exits the reservoir at a second location that is positioned across the first location through sample fluid, on the opposite side of the channel across the width of the channel. Because the first location, or the entry location of the illumination light into the sample fluid, is positioned across the second location, or the exit location of light out of the sample fluid, the average travel path of the illumination light beam through the sample fluid from entry into the reservoir to exit from the reservoir is substantially linear or straight. Therefore, loss of light to scatter can be minimized, such that measurements of the transmitted light reaching the detector can be more repeatable and reliable. The transmitted light 668 enters the second light guide 655, which may be configured similarly to the first light guide 653, such that the light exiting the second light guide is redirected at an off-angle and towards the second lens 656. The second lens 656 may be configured to collimate, focus, or otherwise direct light towards the photodetector 658. The photodetector may be configured to detect an intensity of the transmitted light incident on the detector, converting the incident light into voltage, current, or resistance measurements.

In order to ensure the accuracy of optical measurements made by the optical sensing unit 650, processing unit 640 may be configured to initiate measurements with the optical sensing unit only when a sufficient amount of fluid is present inside the reservoir. For example, the one or more fluid sensors 675 can be interrogated to determine the level of fluid contained inside the reservoir, and the processing unit may be configured with instructions to initiate measurements with the optical sensing unit only when the determined fluid level exceeds a predetermined threshold level of fluid, the threshold level of fluid comprising a level sufficient to cause complete filling of the channel 609. Optionally, in addition, the processing unit may interrogate the one or more reservoir sensors such as the orientation sensors described in reference to sensing reservoir 200 of FIGS. 2A-2C. If the sensing reservoir is determined to be in a substantially inverted orientation, such as during feeding of milk in the reservoir to an infant, the processing unit may be configured not to initiate measurements with the optical sensing unit until the reservoir is determined to be in a substantially upright orientation. Such controlled measurements with the optical sensing unit can ensure that the channel is completely full when the fluid is interrogated with the optical sensing unit, thereby preventing the collection of inaccurate optical data.

In order to optimize the optical measurements made by the optical sensing unit 650 of FIG. 6, the channel 609 may be dimensioned to have a width 611 that is sufficiently small to minimize the scattering of light through the fluid. If the path length of the light through the fluid is short enough, the measurement effectively becomes a non-scattering transmittance measurement, thereby reducing the dynamic range of the measurement. For example, for a channel having a width no greater than about 5 mm, or in a range from about 1 mm to about 5 mm, and therefore providing a path length no greater than about 5 mm or in a range from about 1 mm to about 5 mm, scattered light becomes a less dominant component of the light measured by the photodetector, as the transmissivity of the fluid within the channel becomes a more direct measurement of the solids, fats, and other components of the fluid that are less transparent.

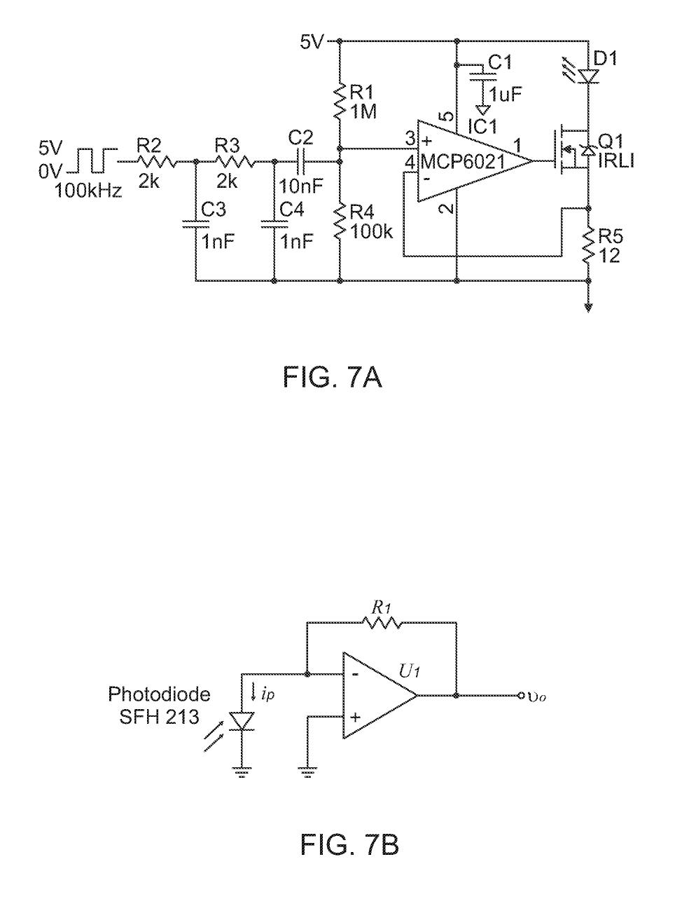

FIG. 7A shows a diagram of an exemplary driver circuit that may be used in any of the embodiments described herein, for an illumination light source for a sensing reservoir having an integrated optical sensing unit. A Pulsed Laser Driver Circuit as shown in FIG. 7A may be used to drive a laser light source of a sensing reservoir as described herein, such as sensing reservoir 400 of FIGS. 4A-4B or sensing reservoir 600 of FIG. 6. The laser light source may be pulsed using the Pulsed Laser Driver Circuit, then the output of the detector of the sensing reservoir sampled. Light and dark currents can be alternatively measured in this way, which can allow the processing unit operably coupled with the optical sensing unit to subtract background light from the measurements to reduce the contribution of ambient light or leakage currents to the measured signal. FIG. 7B shows a diagram of an exemplary amplifier circuit that may be used in this or any other embodiment described herein, for an illumination light source for a sensing reservoir having an integrated optical sensing unit. A Photodiode Transconductance Amplifier Circuit as shown in FIG. 7B may be incorporated into the processing unit to improve the accuracy and repeatability of light measurements with the optical sensing unit.

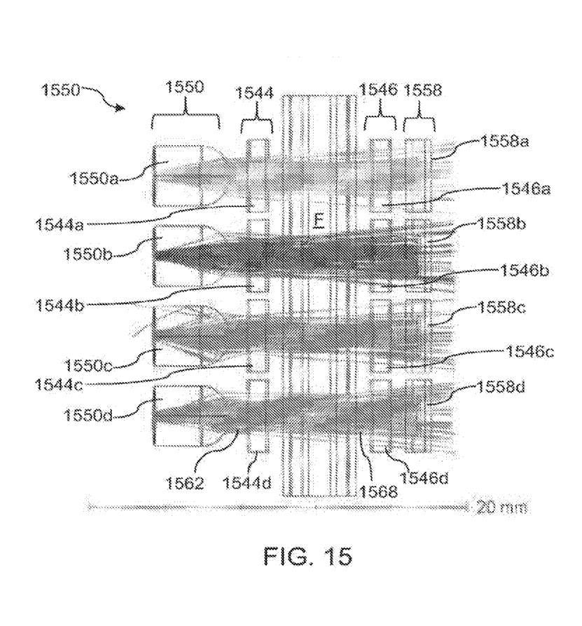

FIG. 14 illustrates another exemplary configuration of an optical sensing unit for measuring light transmitted through a sample fluid contained inside a sensing reservoir, suitable for incorporation with any of the embodiments disclosed herein. Optical sensing unit 1450 may be similar in many aspects to optical sensing unit 650 described with reference to FIG. 6. For example, the optical sensing unit 1450 may comprise one or more illumination light sources 1452 (e.g., LEDs) and one or more detectors 1458 (e.g., silicon or germanium photodetectors), which may be similar in many aspects to illumination light source 652 and detector 658, respectively. Though not shown, the optical sensing unit 1450 may further comprise a processing unit similar to processing unit 640 described with reference to FIG. 6, wherein the processing unit may be in communication with the light source and the detector. The optical sensing unit 1450 may be configured to measure light transmitted through a sample channel 1409 of a sensing reservoir, wherein the sample channel may be formed at the bottom of the reservoir and similar in many aspects to sample channel 609 described with reference to FIG. 6. In optical sensing unit 1450, the light source 1452 is placed laterally adjacent a first side wall 1413a of the sample channel 1409, and the detector 1458 is placed laterally adjacent a second side wall 1413b of the sample channel opposite the first side wall. In this configuration, illumination light beam 1462 emanating from the light source 1452 is directed straight towards the first side wall 1413a without the need for redirection via a light guide. Similarly, the transmitted light 1468 emerging through the second side wall 1413b is directed straight towards the detector 1458. Light therefore enters the reservoir at a first location on the first side wall 1413a of the channel, then exits at a second location on the second side wall 1413b, the second location positioned across from the first location. Optical sensing unit 1450 may further comprise a first bandpass filter 1444 disposed between the light source 1452 and the sample channel 1409, and/or a second bandpass filter 1446 disposed between the sample channel 1409 and the detector 1458. The first bandpass filter may be configured to selectively allow illumination light 1462 within a narrow wavelength range to pass therethrough. Similarly, the second bandpass filter may be configured to selectively allow transmitted light 1468 within the selected narrow wavelength range to pass therethrough. The first and/or second bandpass filters can help ensure that the absorption of light of a specific, target wavelength range is measured. The detector 1458 may be amplified with a moderate gain transimpedance amplifier, and the signal from the detector may be digitized by a microprocessor in communication with the detector. The microprocessor may be further configured to modulate the light source and use digital signal processing detection techniques to optimize the signal-to-noise ratio of the system and to rejected unwanted ambient light.

In any of the embodiments disclosed herein, an optical sensing unit may comprise a plurality of light sources and/or a plurality of detectors. For example, referring to FIG. 6, the optical sensing unit 650 may comprise two or more detectors 658 arranged in an array along the channel 609. Each of the plurality of detectors may be configured to detect light of a predetermined wavelength or range of wavelengths that is different from the wavelength or range of wavelengths configured to be detected by the other detectors. The illumination light source may comprise a single light source or a plurality of light sources. For example, the illumination light source may comprise a single broadband light source or a plurality of narrowband light sources having different central emission wavelengths, such as LEDs. An optical sensing unit can be thus configured to function as a multi-spectral system, capable of interrogating the sample liquid at multiple wavelengths. An optical sensing unit comprising a plurality of narrowband detectors and/or light sources can generate a discrete absorption spectrum of the sample, comprising discrete absorbance measurements at distinct spectral bands. Alternatively, an optical sensing unit comprising a sufficiently large array of detectors, capable of generating sample absorbance measurements over a wide range of wavelengths, can generate a continuous absorption spectrum of the sample.

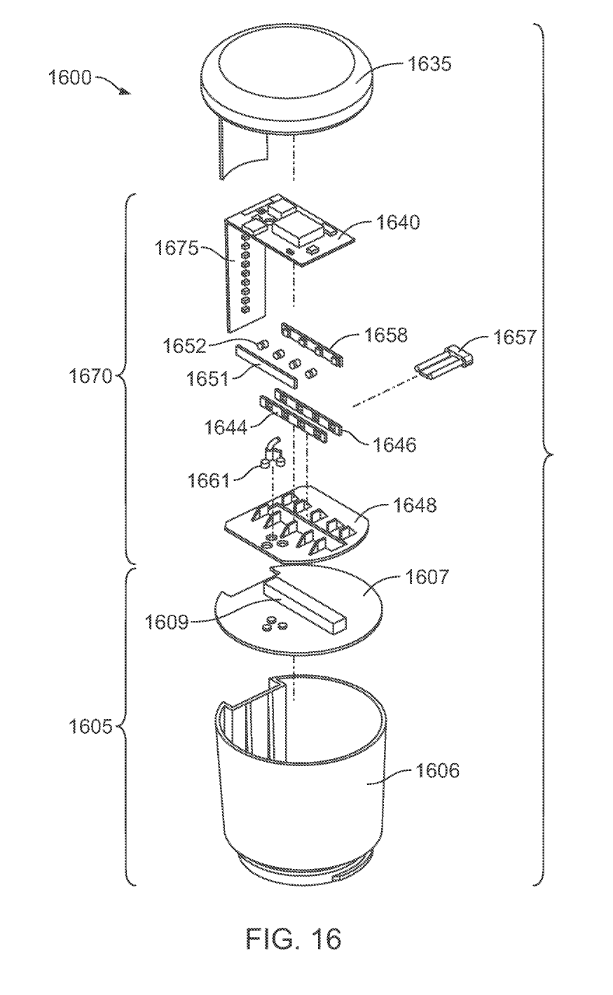

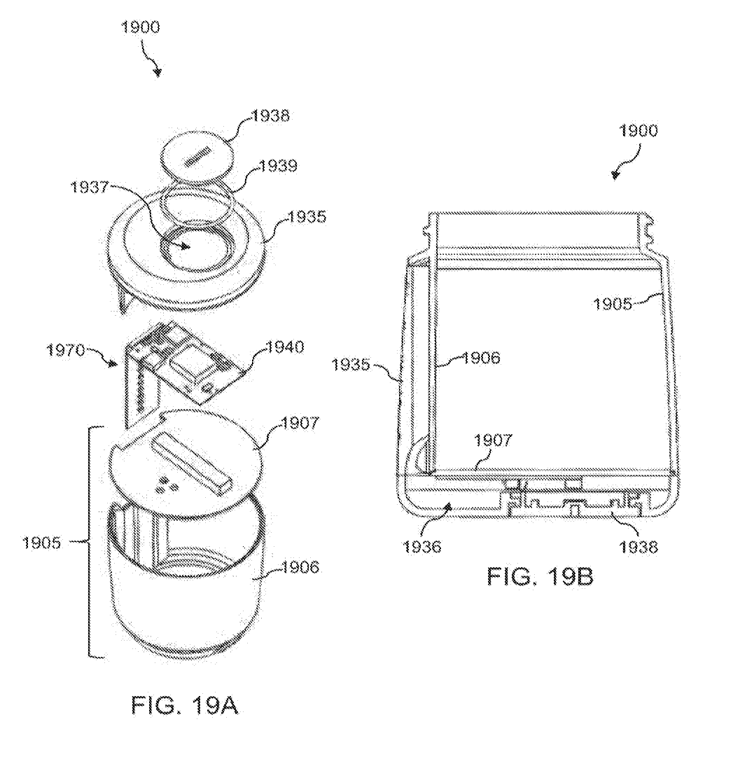

FIG. 15 schematically illustrates an exemplary configuration of an optical sensing unit 1500 for measuring light transmitted through a sample fluid contained inside a sensing reservoir, suitable for incorporation with any of the embodiments disclosed herein. Optical sensing unit 1500 comprises an array 1552 of illumination light sources 1552a, 1552b, 1552c, and 1552d. The optical sensing unit further comprises an array 1558 of detectors 1558a, 1558b, 1558c, and 1558d, wherein each of the plurality of detectors is aligned with each of the plurality of light sources such that the illumination light beams 1562a, 1562b, 1562c, 1562d emanating from each of the plurality of light sources passes through the sample fluid F and hit the detectors 1558a, 1558b, 1558c, and 1558d, respectively. Optical sensing unit 1500 may further comprise a first array 1544 of bandpass filters 1544a, 1544b, 1544c, 1544d, disposed between the array 1550 of light sources and the sample; the optical sensing unit may further comprise a second array 1546 of bandpass filters 1546a, 1546b, 1546c, 1546d, disposed between the sample and the array 1558 of detectors. Each light source/detector pairing can define a distinct measurement channel configured to measure sample absorbance at a distinct central wavelength. In one exemplary configuration of the optical sensing unit 1500, the array of light sources may comprise 4 infrared LEDs, configured to emit illumination light centered about the wavelengths 707 nm, 930 nm, 952 nm, and 1060 nm. The sample absorbance at 707 nm may be used a reference or control measurement, and the sample absorbance at 930 nm, 952 nm, and 1060 nm may be used to determine the relative amount of fats, proteins, and lactose, respectively, as described in further detail herein with reference to FIG. 19. Though FIG. 15 depicts the optical sensing unit 1500 having an array of four light sources and four detectors with matching bandpass filters, the sensing unit may be configured with any number of light sources and detectors suitable for measuring the desired wavelengths or range of wavelengths.

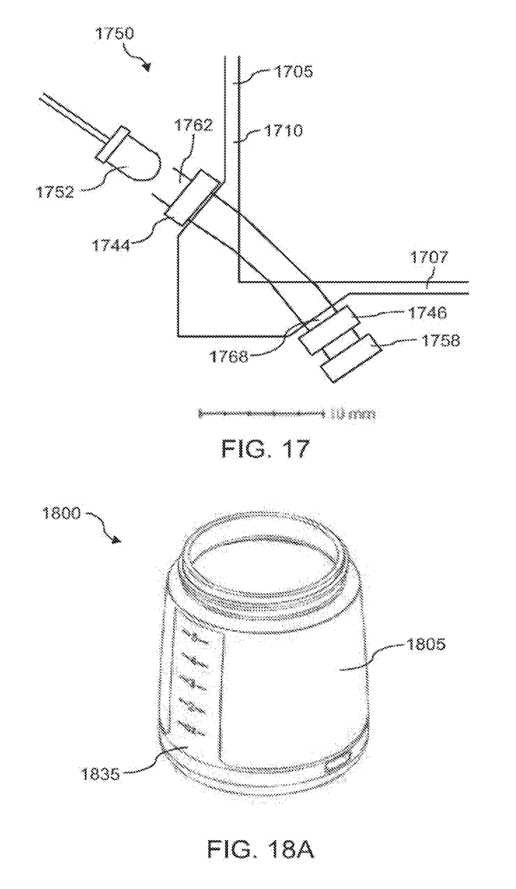

FIG. 16 illustrates an exemplary embodiment of a sensing reservoir 1600 comprising an optical sensing unit as described herein with reference to FIGS. 14 and 15. The sensing reservoir 1600 may comprise a reservoir 1605, a fluid sensing unit 1670 configured to measure various aspects of the sample fluid contained within the reservoir, a housing 1635 configured to enclose the fluid sensing unit within a fluid-tight chamber, and a connector 1657 configured to connect to a power source and/or another computing device (e.g., USB port). The reservoir 1605 may comprise a body portion 1606 and a bottom portion 1607 coupled together to form the sample chamber of the reservoir. The bottom portion 1607 may comprise a bottom channel 1609 of a defined geometry, through which one or more illumination light beams may be directed to measure the absorbance of the illumination light by the sample disposed within the bottom channel. The bottom channel 1609 may be similar in many aspects to the channel 609 described with reference to FIG. 6. The fluid sensing unit 1670 may comprise a fluid level sensor array 1675, a processing unit 1640, and an optical sensing unit comprising a light source array 1652, a detector array 1658, a first bandpass filter array 1644, a second bandpass filter array 1646, and a chassis 1648.

The optical sensing unit may be similar in many aspects to optical sensing units 650, 1450, and 1550 described with reference to FIGS. 6, 14, and 15, respectively, wherein similarly named components may be similar in many aspects. For example, the light sources may comprise narrow-band LEDs, and the detectors may comprise silicon or germanium photodetectors. The LEDs may be supported on a board comprising drive circuitry 1651 for the LEDs; likewise, the detectors may be supported on a board comprising readout and processing circuitry, wherein the light source drive circuitry and/or detector readout circuitry may be in communication with the main processing unit 1640 of the sensing reservoir. The light source array 1652 and detector array 1658 may be arranged to be disposed laterally on either side of the bottom channel 1609 of the reservoir. The first bandpass filter array 1644 may be disposed between the light source array and the bottom channel, and the second bandpass filter array 1646 may be disposed between the bottom channel and the detector array, so as to selectively measure sample absorbance of only the wavelengths of interest. The chassis 1648 may be configured to fit over the bottom channel 1609 and support the lights sources, detectors, and bandpass filters, while appropriately aligning the components with respect to the bottom channel and to one another. The chassis 1648 may be configured block light, so as to reduce interference from ambient light.

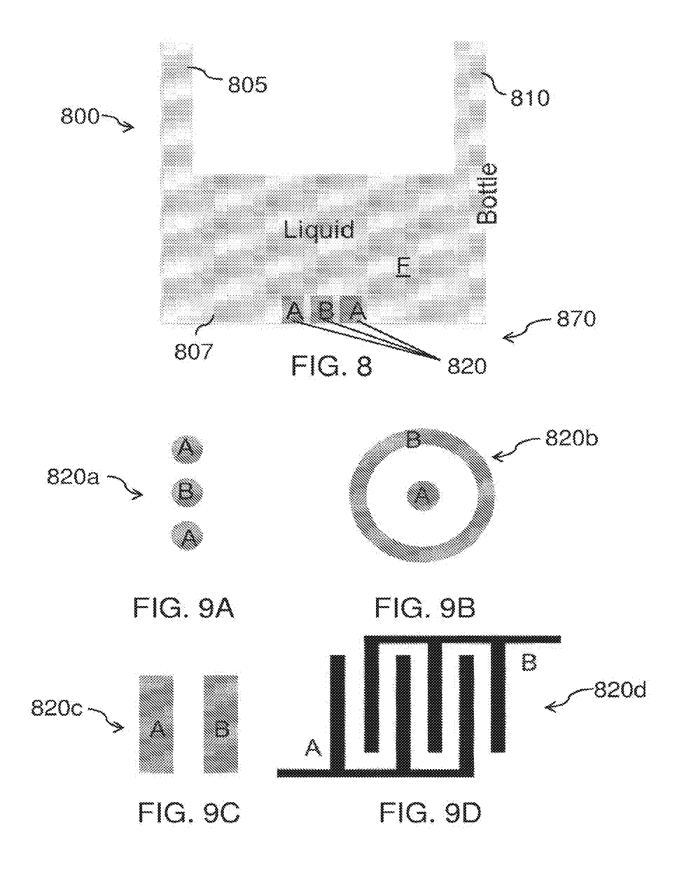

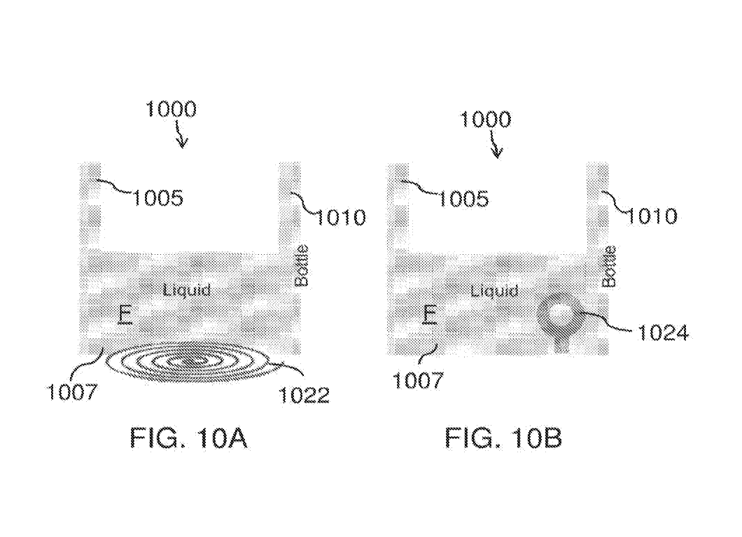

Optionally, the fluid sensing unit 1670 may further comprise a conductance electrode array 1661 configured to measure an electrical conductance of the fluid contained within the reservoir, as described in further detail herein with reference to FIGS. 8-9D. The conductance electrode array 1661 may be mounted to the reservoir bottom portion 1607 as shown.

The processing unit 1640 may be configured with instructions to receive measurement data from the fluid level sensor array 1675, detector array 1658, and conductance electrode array 1661, and process the data, locally store the data, and/or transmit the data to a remote computing device via a wired or wireless connection. In some embodiments, the processing unit may comprise a communications module that enables wireless data transmission, such as Bluetooth™. Alternatively or in combination, data may be transferred through the water-tight connector 1657 to an external data collection card.

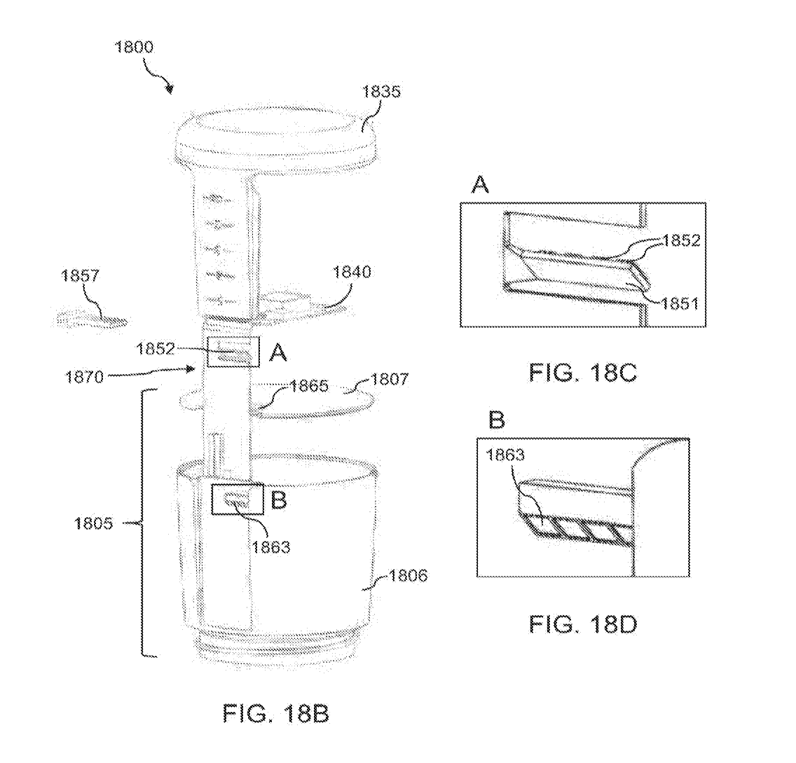

FIG. 17 schematically illustrates another exemplary configuration of an optical sensing unit 1750 for measuring light transmitted through sample fluid contained inside a sensing reservoir, suitable for incorporation with any of the embodiments disclosed herein. Optical sensing unit 1750 may be similar in many aspects to optical sensing units 650, 1450, or 1550 described with reference to FIGS. 6, 14, and 15, respectively. For example, the optical sensing unit 1750 may comprise a light source 1752, a first bandpass filter 1744 coupled to the light source, a detector 1758, and a second bandpass filter 1746 coupled to the detector, wherein these components may be similar in many aspects to similarly named counterparts in the optical sensing units 650, 1450, or 1550. In contrast to the arrangement of the optical components to measure light passed through a thin bottom channel of the reservoir, however, in optical sensing unit 1750, the optical components are arranged to direct the measurement light path through a bottom corner of the reservoir 1705. Light source 1752 may be disposed adjacent a side wall 1710 of the reservoir 1705 not far above the bottom 1707 of the reservoir. The light source may be arranged to direct the illumination light beam 1762 at an oblique, downward angle, such that the light cuts across the bottom corner of the reservoir through the sample fluid F contained within the reservoir, and passes out through the bottom 1707 of the reservoir. Light therefore enters the reservoir at a first location on the side wall of the reservoir, then exits the reservoir at a second location on the bottom wall of the reservoir, the second location positioned across the first location through the reservoir chamber containing the sample fluid. Therefore, the illumination light beam travels from the first location through the sample fluid to the second location along a substantially linear or straight trajectory, so as the minimize the loss of light to scatter or reflection. The body of the reservoir may define appropriately angled seating surfaces for the light source and the detector, to ensure that the first location of light entry and the second location of light exit are aligned along a straight path.

Though not shown in FIG. 17, each light source may be coupled to drive circuitry, detector to read-out circuitry, and the light source and/or detector may be further in communication with a main processing unit for the sensing reservoir configured to receive, process, store, and/or transmit measurement data received from the detector. In some embodiments, each light source may comprise an infrared LED, which in concert with the first bandpass filter can form a narrow band light source. Each detector may comprise a monolithic infrared detector with an internal moderate gain transimpedance amplifier. The signal from the detector may be digitized by a microprocessor. Each detector may be covered with a visible light-blocking and infrared-transmitting plastic. In addition, the microprocessor may be configured to modulate the light sources and use digital signal processing detection techniques to optimize the signal-to-noise ratio of the system and to rejected unwanted ambient light.

Because of the high scattering properties of milk, the total light path of the illumination light traveling through the sample fluid may be estimated as the ensemble average of the highly-scattered trajectories. The scattering properties of the milk may be modeled to determine the optimal range for the detector amplification stage. In order to optimize the optical measurements made by the optical sensing unit, the light source and detector may be arranged such that the path length of light through the fluid that is sufficiently small to minimize the scattering of light through the fluid. For example, the light source and detector may be arranged such that the path length of light traveling through the sample fluid is no greater than about 15 mm, no greater than about 10 mm, or no greater than about 5 mm. The path length of the light can be set by varying the position and orientation of the light source and detector with respect to the reservoir body and bottom.

Though FIG. 17 shows a single light source/detector pair channel, an optical sensing unit having components arranged as shown may have two or more such channels, to enable measurement of sample absorbance at two or more different wavelengths or wavelength ranges. For example, the optical sensing unit may comprise an array of 4 narrow band light sources and an array of 4 detectors aligned with the 4 light sources, wherein the light sources are configured to emit illumination light centered about 4 different wavelengths. The central emission/measurement wavelength of at least a portion of the light channels may be selected based on the known spectral absorption characteristics of analytes of interest. For example, where the analytes of interest include fats, proteins, and lactose, one channel may be configured to measure sample absorbance at 707 nm, to provide a reference or control measurement that is substantially independent of the composition of the three analytes of interest; the remaining three channels may be configured to measure sample absorbance at 930 nm, 952 nm, and 1060 nm, to determine the relative amount of fats, proteins, and lactose, respectively, as described in further detail herein with reference to FIG. 19. Having three measurement targets and four signals enables the use of optimized least-squared fit data analysis methods to provide well-defined target estimates.

The arrangement of optical components in the optical sensing unit 1750 utilizes the already existing geometry of the reservoir 1705, the perpendicularity of the reservoir side wall and the reservoir bottom, to create the sample channel. Such an arrangement can help not only lower the cost of the system by simplifying the design of the system, but it can also help improve the performance of the system by reducing the risk that air may get trapped in the sample channel and thus impede accurate measurement of the sample fluid, and by improving the cleanability of the reservoir.

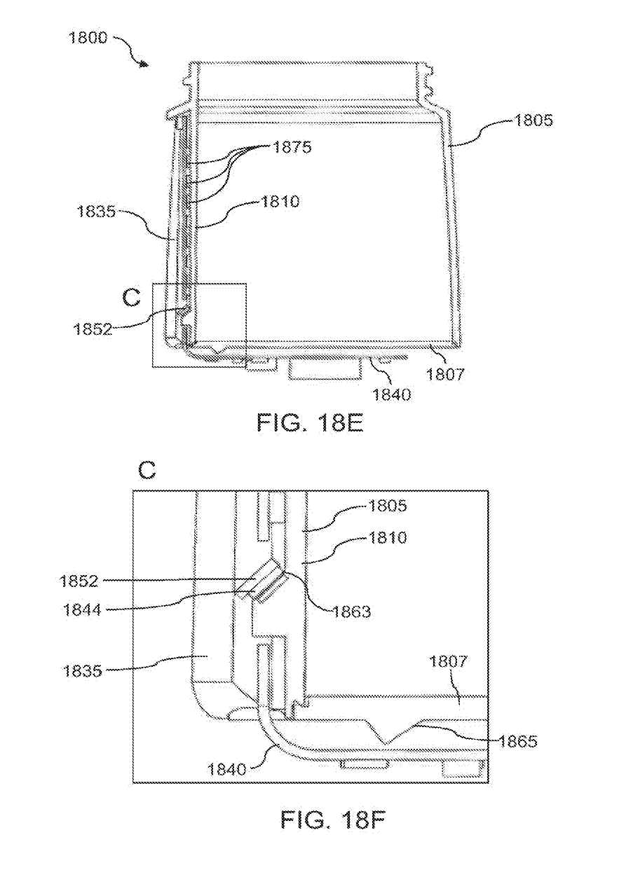

FIGS. 18A-18F illustrate an exemplary embodiment of a sensing reservoir 1800 comprising an optical sensing unit as described herein with reference to FIG. 17. FIG. 18A is an isometric view of the sensing reservoir 1800, FIG. 18B is an exploded view, FIG. 18C is a detail view of section A of FIG. 18B, FIG. 18D is a detail view of section B of FIG. 18B, FIG. 18E is a side cross-sectional view, and FIG. 18F is a detail view of section C of FIG. 18E.

The sensing reservoir 1800 may comprise a reservoir 1805, a fluid sensing unit 1870 configured to measure various aspects of the sample fluid contained within the reservoir, a housing 1835 configured to enclose components of the fluid sensing unit within a fluid-tight chamber, and a connector 1857 configured to connect to a power source and/or another computing device (e.g., USB port). The reservoir 1805 may comprise a body portion 1806 and a bottom portion 1807 coupled together to form the sample chamber of the reservoir. The body portion 1806 may comprise an input light guide 1863, and the bottom portion 107 may comprise an output light guide 1865. The fluid sensing unit 1870 may comprise a fluid level sensor array 1875, a processing unit 1840, and an optical sensing unit comprising a light source array 1852 and a detector array (not shown). The optical sensing unit may further comprise a first bandpass filter array 1844 (best seen in FIG. 18F) coupled to the light source array, and/or a second bandpass filter array (not shown) coupled to the detector array, the bandpass filters configured to selectively transmit a narrow spectral band of light therethrough, so as to form a plurality of measurement channels each configured to detect sample absorbance over a distinct spectral band.

The light source array, detector array, and bandpass filter arrays may be similar in many aspects to similarly named components described herein with respect to various embodiments, for example the optical sensing unit 1750 shown in FIG. 17. For example, the light sources may comprise narrow-band LEDs, and the detectors may comprise silicon or germanium photodetectors. The LEDs may be supported on a board comprising drive circuitry for the LEDs, and the detectors may be supported on a board comprising readout and processing circuitry, wherein the light source drive circuitry and/or detector readout circuitry may be in communication with the main processing unit 1840 of the sensing reservoir. The light source array and detector array may be arranged to direct the measurement light path through a bottom corner of the reservoir 1805. Light source array 1852 may be disposed adjacent a side wall 1810 of the reservoir 1805 not far above the bottom 1807 of the reservoir. The light source array may be arranged to direct the illumination light beam at an oblique, downward angle, such that the light cuts across the bottom corner of the reservoir through the sample fluid contained within the reservoir, and passes out through the bottom 1707 of the reservoir towards the detector array. For example, as best seen in FIGS. 18C, 18D, and 18F, the light source array 1852 may be supported by an input light guiding structure 1863 disposed on the side wall 1810 of the reservoir, wherein the input light guide 1863 positions the light source array at an appropriate angle to direct the illumination light towards the bottom 1807 of the reservoir where the detector array is arranged. The output light guiding structure 1865, disposed on the bottom 1807 of the reservoir, may direct the light transmitted through the sample fluid onto the detector array. Optionally, the detector array and/or a second array of bandpass filters may be mounted onto the output light guide 1865.

Optionally, the fluid sensing unit 1870 may further comprise a conductance electrode array configured to measure an electrical conductance of the fluid contained within the reservoir, as described in further detail herein with reference to FIGS. 8-9D. The conductance electrode array may be mounted to the reservoir bottom portion 1807, for example.

The processing unit 1840 may be configured with instructions to receive measurement data from the fluid level sensor array 1875, detector array 1858, and/or a conductance electrode array, and process the data, locally store the data, and/or transmit the data to a remote computing device via a wired or wireless connection. In some embodiments, the processing unit may comprise a communications module that enables wireless data transmission, such as Bluetooth™. Alternatively or in combination, data may be transferred through the water-tight connector 1857 to an external data collection card.

FIGS. 19A-19B illustrate an exemplary embodiment of a sensing reservoir 1900, aspects of which may be suitable for incorporation with any embodiment of a sensing reservoir as described herein. FIG. 19A is an exploded view, and FIG. 19B is a side cross-sectional view of the sensing reservoir 1900. Sensing reservoir 1900 may be similar in many aspects to various embodiments of a sensing reservoir disclosed herein. For example, the sensing reservoir 1900 may comprise a reservoir 1905, a fluid sensing unit 1970, and a housing 1935, similar in many aspects to similarly named components described with reference to other embodiments of a sensing reservoir. The fluid sensing unit 1970, which may include one or more of a fluid level sensor array, optical sensing unit, or electrical conductance sensor array, as well as processing unit 1940 in communication with the one or more sensors of the fluid sensing unit, may be housed within an enclosed, fluid-tight chamber 1936, in order to protect the various sensors and/or electrical components from fluid ingress and damage. As shown in FIG. 19B, the fluid-tight chamber 1936 may be formed between an interior wall of the housing 1935 and an exterior wall of the reservoir body 1906 and/or the reservoir bottom 1907. Optionally, the housing may be shaped to form an opening 1937 to allow access to one or more of the components encased in the chamber 1936. The opening 1937 may be reversibly sealed with a cover 1938, and a sealing member 1939 may optionally be added to the assembly to ensure a fluid-tight seal between the housing 1935 and the cover 1938. For example, the sealing member 1939 may be an o-ring dimensioned to fit within a corresponding o-ring groove formed in the housing 1935 adjacent the opening 1937, and configured to be held compressed into the o-ring groove by the cover 1938 to form a fluid-tight seal therebetween. The opening 1937 may be located at any suitable location of the housing 1935, such as at the bottom of the housing 1935 as shown in FIGS. 19A and 19B. The opening 1937 can allow a user to easily replace one or more components housed within the chamber 1936, such as a battery configured to provide power to the fluid sensing unit. The fluid-tight cover 1938 can help prevent damage to any permanently encased components and/or electrical connections disposed in the chamber 1936, in particular during cleaning of the sensing reservoir 1900.

Optionally, in any of the embodiments described herein, an optical sensing unit such as the optical sensing unit 450 of FIGS. 4A-4B, optical sensing unit 650 of FIG. 6, or any other optical sensing unit as shown and described herein, may be configured to generate calibrated measurements rather than absolute irradiance measurements, in order to reduce the susceptibility of the measurements to errors related to mechanical tolerances of the system. The optical sensing unit may be used to obtain calibration measurements, for example of known standards, and the actual sample measurements may be compared to the calibration measurements to generated relative transmission or relative scattering. The calibration measurements may be stored on-board the local processing unit of the optical sensing unit, and/or they may be stored on a remote processing unit in communication with the local processing unit. Such a calibration process can allow the use of a defocused laser light source, and/or reduce the tolerance requirements for various mechanical components of the sensing unit, such as the size and/or position of any of the lenses, light guides, or detector, or the dimensions of portions of the reservoir such as the channel.

In any of the embodiments disclosed herein, an optical sensing unit may be configured to interrogate a specific wavelength or range of wavelengths of interest. The wavelengths to be interrogated by the optical sensing unit may be selected based on the absorption spectra of analytes of interest, such as fats, lipids, carbohydrates, proteins, glucose, lactose, salts, or other organic or inorganic molecules, compounds, or constituents of the sample contained in the reservoir such as breast milk. Many of these analytes have been shown to have unique signatures in their near-infrared absorbance spectra. Thus, measurements of light of a select wavelength transmitted through the sample fluid may be used to derive the relative amount of a component of the sample fluid corresponding to the wavelength. An optical sensing unit as described herein may be configured to measure the absorption of light by the sample in the near infrared (e.g., 0.7-2.0 um) and/or the mid-infrared region (e.g., 2.0-7.0 um).

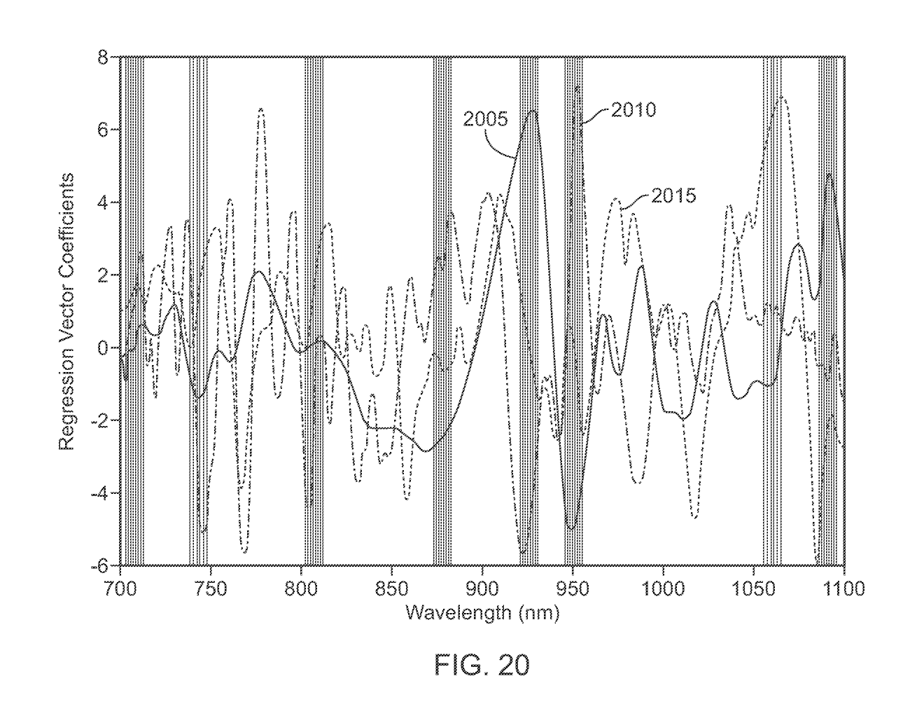

FIG. 20 is a graph of regression vector data of the near-infrared absorption spectra of fat, total protein, and select lactoses. Graph 2005 represents the data for fat, graph 2010 represents the data for protein, and graph 2015 represents the data for lactose. Wavelengths suitable for distinguishing fat, total protein, and select lactoses may include spectral bands where one graph is high and the other graphs are low. For example, for fat determination, bands around 930 nm and 1092 nm may be well-suited; for protein determination, bands at around 952 nm and 880 nm may be well-suited, and 760 nm, 776 nm, and 1034 nm may be other good options; for lactose determination, bands at around 1060 nm and 745 nm may be well-suited. Spectral bands at around 707 nm and 806 nm, at which absorption is largely independent of the relative amounts of fat, protein, and lactose, may be well-suited to serve as control or reference measurements. Work in relation to embodiments suggests that wavelengths particularly well-suited for the detection of fats, proteins, and lactose may be 707 nm (control), 930 nm (fats), 952 nm (proteins), and 1060 nm (lactose). Other suitable spectral bands may also be identified by measuring the absorption of the sample fluid of interest across a wide range of wavelengths in the infrared-NIR region. For example, samples of unmodified breast milk as well as breast milk modified with known quantities of the target analytes (e.g., fats, proteins, lactose) may be measured over the wavelength range of about 650 nm to about 1100 nm using a reference absorbance spectrometer, and the measurement data analyzed to identify a small number of spectral bands suitable for the detection of the target analytes. Measurements of the absorption/transmission of light through the sample fluid at the selected spectral bands may be used to derive the relative amounts of the target analytes in the sample fluid. As described herein, the total caloric content of a sample of breast milk may be calculated based on the relative amounts of fats, proteins, and lactose in the sample, using the following equation: Caloric content=[% Fat*Fat calories]+[% Protein*Protein calories]+([% Lactose*Lactose calories]. Such a calculation may be performed by a processing unit in communication with the optical sensing unit, as described herein.

In any of the embodiments disclosed herein, an optical sensing unit may be configured to interrogate the sample fluid at multiple different wavelengths or wavelength ranges, thus producing multi-spectral absorbance data of the sample fluid. For example, as described herein with reference to several embodiments, an optical sensing unit may comprise a plurality of measurement channels each comprising a pair of narrow band light source and detector, where measurement data from each of the plurality of channels may be processed to generate a discrete absorption spectrum of the sample, comprising discrete absorbance measurements at distinct spectral bands. Alternatively, a micro-spectrometer capable of producing a continuous absorption spectrum of the sample in the NIR-IR range may be incorporated with a sensing reservoir as described herein. To enable generation of a continuous absorption spectrum, the micro-spectrometer may comprise a large array of detectors, capable of generating sample absorbance measurements over a wide range of wavelengths. The detector array may be coupled with a matching array of optical filters, or a dispersive optical element (e.g., diffraction grating, prisms) in a miniaturized form factor, which enables splitting of the incident light beam onto well-defined areas of the detector array. The array of detectors may be combined with a matching array of narrow band illumination light sources, or to a broadband light source. The light source(s) and detectors of such a micro-spectrometer system may be arranged within the sensing reservoir in any configuration as described herein, for example as shown and described in reference to FIGS. 3-6 and 14-18F. Some micro-spectrometer systems suitable for incorporation with a sensing reservoir as disclosed herein are described in the following publications: U.S. Pat. Nos. 9,377,396; 9,395,473; US Patent Publication No. US20140061486.

In some embodiments, the processing unit may be configured with instructions to process raw measurement data generated by the photodetector to produce an absorption spectrum of the sample substance. In some embodiments, the processing unit may be configured to instructions to send raw or processed measurement data to a remote processing unit (e.g., of a remote computing device in communication with the local processing unit via a wired or wireless connection), wherein the remote processing unit may be configured with instructions to generate the absorption spectrum of the sample substance. The local processing unit and/or a remote processing unit in communication with the local processing unit may be further configured with instructions to analyze the raw measurement data and/or the absorption spectrum of the sample to determine a compositional aspect of the sample, such as the relative content of fats, proteins, or any other component of interest. In some embodiments, the local processing unit and/or a remote processing unit may be configured with instructions to pre-process the raw measurement data prior to the generation of an absorption spectrum or determination of sample composition. For example, pre-processing may include signal processing to filter out or reduce signal contributions from unwanted ambient light.

A fluid sensing unit of a sensing reservoir as disclosed herein may comprise one or more sensors configured to measure a conductivity of the fluid contained inside the reservoir. For example, as described in further detail herein, a fluid sensing unit may comprise one or more electrodes or inductor coils. The conductivity of the fluid may indicate the relative amounts of one or more components of the fluid. For example, the conductivity of a breastmilk sample can indicate the salinity of the milk, since soluble salt in the milk will generally increase the conductivity of the milk. Other constituents of breastmilk may impact the conductivity measurements to varying degrees. For example, increasing fat content in the milk may decrease the conductivity of the milk, while lactose may have no substantial impact on the conductivity of the milk. The information relating the composition of the milk, derived from conductivity measurements, may be used to provide feedback and clinical recommendations relating to the health of the mom as well as the nutrition provided to the infant via the breastmilk.

FIG. 8 illustrates an exemplary configuration of a sensing reservoir 800 comprising one or more electrodes 820 for measuring the conductivity of the fluid contained inside the reservoir. The sensing reservoir 800 may be used in combination with other components of a pumping device, such as the various components of pumping device 100 shown in FIG. 1. The sensing reservoir 800 may be similar in one or more aspects to the sensing reservoir 200 shown in FIGS. 2A-2B, and may comprise one or more features or elements described in reference to the sensing reservoir 200. For example, the sensing reservoir 800 may comprise a reservoir 805 and a fluid sensing unit 870, which may be similar in many aspects to reservoir 205 and fluid sensing unit 270. For example, the fluid sensing unit 870 may comprise one or more fluid sensors configured to measure an amount of the fluid contained inside the reservoir. Optionally, the fluid sensing unit 870 may further comprise an optical sensing unit as in any optical sensing unit disclosed herein, such as optical sensing unit 650 described with reference to FIG. 6.

The fluid sensing unit 870 may further comprise one or more electrodes 820 configured to measure the conductivity of the fluid F. The fluid sensing unit may further comprise driving circuitry (not shown) operably coupled with the one or more electrodes, configured to charge or drive an electrode and detect the current flowing between the two terminals of the electrode, or between a drive electrode (terminal A) and a sense electrode (terminal B) of an array of electrodes. The measured current can indicate the conductivity of the fluid F inside the reservoir, which may in turn indicate one or more properties of the fluid F. As described in further detail herein, the measurements generated by the electrodes may be further adjusted based on output from other sensors of the fluid sensing unit, such as a temperature sensor and/or optical sensor.

The one or more electrodes 820 may be disposed in any location of the reservoir 805 appropriate for measuring the conductivity of the fluid F in the reservoir, such as embedded within the wall 810 or bottom 807 of the reservoir. The electrodes 820 may be embedded in the reservoir such that the electrodes are in contact with the fluid F inside the reservoir. In many embodiments, the fluid sensing unit may comprise a plurality of electrodes arranged to increase the coupling between the sense and drive electrodes.

FIGS. 9A-9D show exemplary configurations of electrodes suitable for incorporation with the sensing reservoir 800 of FIG. 8. Specifically, the figures show various topologies of amperometric electrode arrays, wherein FIG. 9A shows a plurality of electrodes 820a arranged side by side, FIG. 9B shows a plurality of coaxial electrodes 820b, FIG. 9C shows a plurality of parallel plate electrodes 820c, and FIG. 9D shows a plurality of interdigitated electrodes 820d. Each of the electrodes of an electrode array may be molded or inserted into the wall or bottom of the reservoir. The driving circuitry coupled to the electrodes may be configured to drive either terminals A or B as shown in FIGS. 9A-9D.

Optionally, one or more electrodes of the sensing reservoir may be futher configured to detect concentrations of certain constituents or components of the fluid F contained inside the reservoir via electrochemical analysis. For example, an oxidizing reagent may be coated on electrodes embedded in the wall of the reservoir, wherein the electrodes make contact with the fluid contained within the reservoir. The oxidizing reagent can interact with the target constituent to result in oxidation at an anode plated with platinum or other suitable material. The oxidation reaction can create an amperometric response that is proportional to the concentration of the particular constituent being measured. In some embodiments, an electrodes may be plated with a glucose oxidase enzyme, which can interact with glucose in the sample fluid to create hydrogen peroxide. The hydrogen peroxide can in turn oxidize at the anode plate coated with platinum, generating a measurable amperometric response. Such electrochemical analysis can be used to detect concentrations of various constituents of interest within the sample fluid, such as alcohol, lactose or glucose.

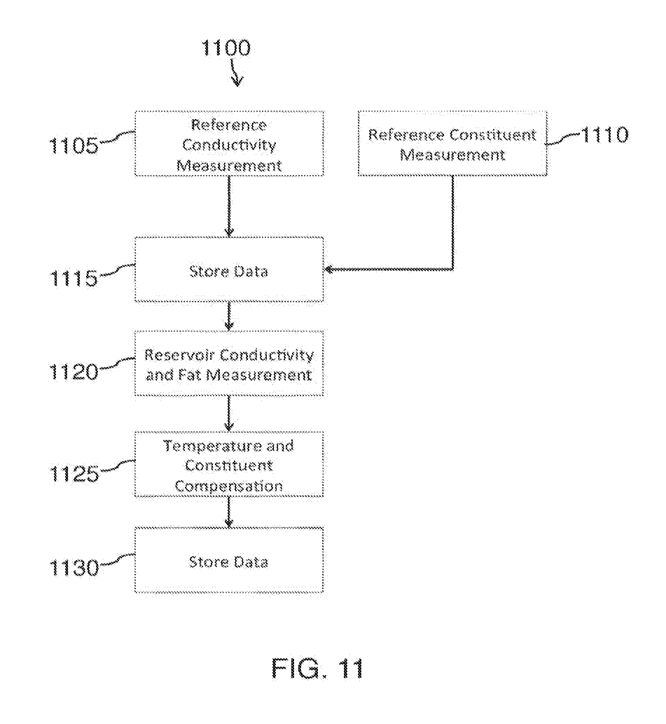

FIGS. 10A and 10B illustrate exemplary configurations of a sensing reservoir 1000 comprising one or more inductors for measuring the conductivity of the fluid F contained inside the reservoir. The sensing reservoir 1000 may be used in combination with other components of a pumping device, such as the various components of pumping device 100 shown in FIG. 1. The sensing reservoir 1000 may be similar in one or more aspects to the sensing reservoir 200 shown in FIGS. 2A-2B, and may comprise one or more features or elements described in reference to the sensing reservoir 200. For example, the sensing reservoir 800 may comprise a reservoir 1005 and a fluid sensing unit 1070, which may be similar in many aspects to reservoir 205 and fluid sensing unit 270. For example, the fluid sensing unit 1070 may comprise one or more fluid sensors configured to measure an amount of the fluid contained inside the reservoir. Optionally, the fluid sensing unit 1070 may further comprise an optical sensing unit as in any optical sensing unit disclosed herein, such as optical sensing unit 650 described with reference to FIG. 6.

The fluid sensing unit 1070 may further comprise one or more inductors configured to measure the conductivity of the fluid F. For example, as shown in FIG. 10A, the fluid sensing unit 1070 may comprise an inductor coil 1022 disposed underneath the fluid F, outside the reservoir and adjacent the bottom wall 1007. The fluid sensing unit may further comprise circuitry (not shown) operably coupled with the inductor coil 1022. The inductor coil can form part of a circuit that measures the inductance L of the coil, and the resonance impedance or parallel resistance Rₚ. The circuit may comprise, for example, an LC oscillator, which can produce a periodic signal at a frequency proportional to the inductance L and capacitance C of the system. As AC current passes through the inductor coil 1022, it can induce an eddy current in the conductive solution F, which, in turn, can change the self-resonant frequency of the oscillator. This change in frequency, which can be proportional to the conductivity of the fluid F, may be detected by the circuitry.

Alternatively, as shown in FIG. 10B, the fluid sensing unit 1070 may comprise a toroidal inductor pair 1024 comprising a first toroidal coil and a second toroidal coil. The toroidal inductor pair may be embedded in or coupled to a bottom wall 1007 or side wall 1010 of the reservoir 1005, such that it is at least partially suspended in the fluid F contained inside the reservoir. The fluid sensing unit may further comprise circuitry (not shown) operably coupled with the toroidal inductor pair 1024. An AC current may be passed through the first toroidal drive coil, which can induce a first current in the fluid F. The first induced current can in turn induce a second current in the second toroidal coil, which may be detected by the circuitry and converted to a voltage. The amount of the second current induced in the second toroidal coil may be proportional to the conductivity of the fluid.

Conductivity measurements made by conductivity sensors as disclosed herein, such as one or more electrodes or inductors as shown and described with reference to FIGS. 8-10B, may be calibrated to a reference standard. A calibration measurement may be taken prior to measurement of the sample fluid of interest, wherein the calibration measurement may comprise measurement of a reference fluid having a known conductivity and temperature. A calibration measurement can be taken using a fluid-filled reservoir at the time of manufacture, for example. Each sensing reservoir may be calibrated individually by measuring each fluid-filled reservoir, or many sensing reservoirs may be calibrated using a single calibration measurement taken with one fluid-filled reservoir, if the calibration measurement is determined to be suitable for application to other reservoirs (e.g., properties of reservoir and sensor components are sufficiently similar to yield comparable calibration measurements).

The conductivity measurements may be further corrected for temperature-dependent effects, as they can be strongly affected by the temperature of the sample fluid. The fluid sensing unit may comprise one or more temperature sensors configured to measure one or more of ambient temperature, temperature of the fluid contained in the reservoir, and/or temperature of one or more components of the sensing reservoir. The temperature measurements made by the one or more temperature sensors may be provided to a processing unit of the fluid sensing unit, where the temperature measurements may be applied to correct the measurements made by conductivity sensors for temperature-dependent effects. Optionally, measurements made by other sensors of the fluid sensing unit (e.g., capacitive sensors, optical sensors, etc.) may also be processed to have any temperature-dependent effects reduced. For example, temperature may affect measurements made by an optical sensing unit as described herein, due to effects on the reservoir material and/or various components of the optical sensing unit. Therefore, the output of the optical sensing unit may be adjusted in response to temperature data generated by the temperature sensors.

To compensate the conductivity measurements for temperature-induced effects, a first order compensation may be applied at the sensor read-out circuitry. Compensation of the measured signal at the read-out circuitry allows the conductivity measurement to be taken at any temperature, wherein a compensation calculation may be subsequently applied to the measured signal to estimate what the measured conductivity would be at a reference temperature. The first order temperature coefficient, a, of the correction may be determined prior to manufacture of the sensing reservoir, and stored in a computer-readable memory of the sensing reservoir operably coupled with the sensor-associated circuitry (e.g., memory on board a processing unit 240 as shown in FIG. 2C). An example of an equation that may be used to apply the first order temperature compensation is shown in Eq. 1:

$${C}_{{T}_{ref}} = \frac{{C}_{Tmeas}}{1 + α ( {T}_{meas} - {T}_{ref} )} \qquad ( Eq . 1 )$$

wherein C_{Tref} is the estimated conductivity of the sample at a reference temperature, C_{Tmeas} is the conductivity of the sample at the measured temperature, a is the first order temperature coefficient, Tₘₑₐₛ is the measured temperature, and T_{ref} is the reference temperature.

Additionally, another correction may be applied to compensate for the effects of a particular component of the sample fluid on the temperature-induced effects on conductivity measurements. For example, the amount of fat in the sample fluid may affect the conductivity of the sample fluid, and temperature may affect the fat-induced changes in measured conductivity. To correct for the combined effects of temperature and fat level on the measured conductivity, a first-order fat coefficient, β, may be determined prior to manufacture of the sensing reservoir, and also stored into the computer-readable memory of the sensing reservoir. An example of an equation that may be used to apply the first order temperature compensation including a correction for fat is shown in Eq. 2:

$${C}_{{T}_{ref}} = \frac{{C}_{Tmeas}}{1 + α ( {T}_{meas} - {T}_{ref} ) + β ( {X}_{Meas} - {X}_{ref} )} \qquad ( Eq . 2 )$$

wherein Xₘₑₐₛ is a measurement of the fat component of the sample at the measurement temperature, and X_{ref} is a measurement of the fat component of the sample at the reference temperature. For example, the measurements of the fat component may comprise optical measurements of the sample as disclosed herein. Similar corrections may be applied for other components of the sample fluid that may affect the temperature-induced effects on conductivity measurements.

Temperature-induced effects on conductivity measurements made with inductors as disclosed herein may be also be at least partially attributed to the temperature coefficients of the material of the inductor coil and/or the sample material being measured. If the temperature coefficients of the materials are known, a calculation can be performed to adjust the measured value for these material-induced effects. As shown in Eq. 3, a first-order compensation may be applied to the measured parallel resistance to correct for the contributions of inductor material properties to the temperature-induced effects on conductivity measurements:

$${Rp}_{Tref} = \frac{{Rp}_{Tmeas}}{1 + α ( {T}_{meas} - {T}_{ref} )} \qquad ( Eq . 3 )$$

wherein Rp_{Tref} is the estimated inductance measurement of a sample at the reference temperature, Rp_{Tmeas} is the inductance measurement of the sample at the measurement temperature, a is the temperature coefficient of the material inductor material, Tₘₑₐₛ is the measurement temperature, and T_{ref} is the reference temperature. In the case of an inductor comprising a copper coil, the temperature coefficient of the material, a, may be about 17 ppm/degree Celsius (° C.).

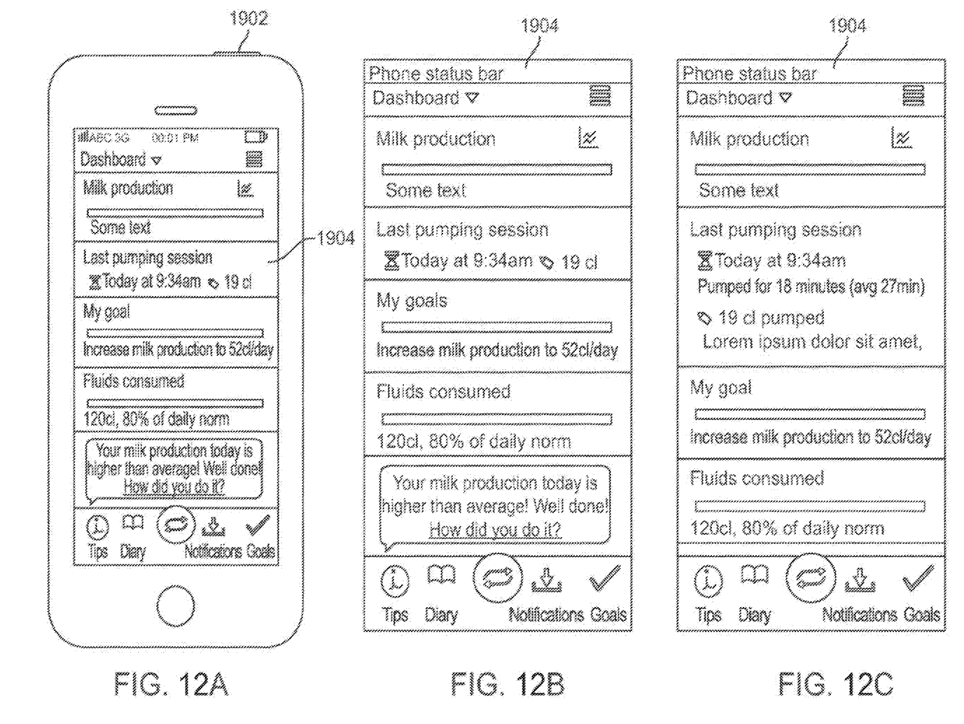

FIG. 11 shows an exemplary method 1100 of measuring the conductivity of a fluid contained in a sensing reservoir as disclosed herein.

In step 1105, the conductivity of a reference fluid at a reference temperature (e.g., 20° C.) may be measured using one or more conductivity sensors of a sensing reservoir as described herein, to yield reference conductivity measurements C_{Tref}. Measurements made using inductor coils as described herein may be converted into parallel resistance Rp_{Tref}.

In step 1110, an additional measurement may be taken of the reference fluid, to measure a specific component or constituent of the reference fluid (e.g., fat). For example, the additional constituent measurement may be taken using a optical sensing unit as described herein.

In step 1115, the reference conductivity measurements (C_{Tref} or Rp_{Tref}) and/or the additional constituent measurements (X_{ref}) may be stored onto a computer-readable memory of the sensing reservoir. The stored reference conductivity measurements may be used as reference in subsequent conductivity measurements of sample fluids. Further, the reference conductivity measurements may be used to determine the temperature coefficient α and/or constituent coefficient β, which may also be stored onto the memory of the sensing reservoir.

In step 1120, the conductivity of a sample fluid contained inside the sensing reservoir is measured at the measurement temperature using one or more conductivity sensors as described herein. For example, a current may be measured and converted to voltage that relates to conductivity, C_{Tmeas}. The measured value may be converted into parallel resistance Rp_{Tmeas}. An additional measurement may be taken of a particular constituent of the sample fluid, such as fat, to yield the constituent measurement Xₘₑₐₛ. The constituent measurement may be obtained using an optical sensing unit as described herein, for example.

In step 1125, the sample fluid conductivity measurements may be corrected for temperature- and/or sample constituent-dependent effects. Tempeature compensation may be applied using a linear compensation model as described in Eqs. 1 or 3, wherein the temperature coeffecient, α, may be pre-determined and stored in the memory of the sensing reservoir. Additionally, compensation for a specific constituent X of the sample fluid, such as fat, may be applied using a linear model as described in Eq. 2, wherein the constituent coefficient, β, may also be pre-determined and stored in the memory of the sensing reservoir.

In step 1130, the corrected measurement data is stored in the memory of the sensing reservoir and/or transmitted to another computing device in communication with the fluid sensing unit, such as a smartphone or a tablet.

One or more steps of the method 1100 may be performed with circuitry as described herein, for example, a processing unit of a sensing reservoir as described herein. The circuitry may be programmed to provide one or more steps of the method 1100, and the program may comprise programmed instructions stored on a computer readable memory. A person of ordinary skill in the art will recognize many variations of the method 1100, based on the teachings described herein. For example, the steps may be completed in a different order. One or more steps may be added or omitted. Some of the steps may comprise sub-steps. Many of the steps may be repeated as often as necessary or desired.

A sensing reservoir in accordance with embodiments may comprise any combination of fluid sensors as described herein. For example, a fluid sensing unit of a sensing reservoir as disclosed herein may include one or more of capacitive sensors, optical sensors, or conductivity sensors. The sensing reservoir may comprise one or more processing units operably coupled to the one or more sensors of the fluid sensing unit, wherein a processing unit may be configured to control interrogation by the one or more sensors and/or receive, store, analyze, and/or transmit to another computing device the measurement data generated by the one or more sensors. The fluid sensing unit may comprise a single processing unit may be configured to control various different sensors of the fluid sensing unit, or the fluid sensing unit may comprise a plurality of processing units each configured to control a sensor or group of sensors configured to measure a specific property of the fluid (e.g., capacitance, conductivity, optical transmission/scattering/absorption, etc.).

Sensors of a sensing reservoir in accordance with embodiments may be configured to selectively to collect data only during times the reservoir is determined to be in a specific state or orientation, in order to help conserve power as well as reduce the collection of unusable (e.g., excessively noisy) data points. For example, as described in further detail herein, fluid level sensors may be configured to collect data only when the reservoir is determined to be in a stable filling state or draining state without excessive detected motion, or when the reservoir is determined to have exited “sleep” state and entered into an active operation state such as a filling state or a draining state. Optical or electrical conductivity sensors as described herein may be configured to collect data only when the reservoir is determined to be in a filling state (e.g., based on data from orientation sensors and/or proximity sensors indicating the connection of the reservoir to a pumping device), to ensure that sample fluid is present in the measurement path for the optical or conductivity sensors. To control the selective interrogation of a specific sensor, the main processing unit of the sensing reservoir may be configured with instructions to activate or deactivate the sensor in response to a determination that the reservoir is in a suitable or unsuitable state for measurement with the sensor.

In embodiments of a sensing reservoir comprising a combination of fluid quantity sensors (e.g., capacitive sensor array) and fluid composition sensors (e.g., optical/spectral sensors, electrical conductivity sensors), both the quantity and composition information produced by the sensors may be used to generate an output of interest. For example, if the output of interest is the total caloric content of a sample of breast milk contained in the reservoir, and the sensing reservoir comprises fluid level sensors and an optical sensing unit configured to determine an absorbance spectrum (either discrete or continuous) of the sample milk, the fluid level data produced by the fluid level sensors and the fluid composition data produced by the optical sensing unit may be combined to generate the desired output of total caloric content of the sample milk. Determination of the output of interest may be performed by the local processing unit onboard the sensing reservoir, and/or it may be performed by a remote processing unit in communication with the local processing unit. For example, in some embodiments, the local processing unit may be configured to transmit raw or partially processed sensor data to the remote processing unit, and the remote processing unit may be programmed with instructions to generate the desired output based on the sensor data input. In some embodiments, the local processing unit may be configured to generate the desired output locally, and transmit the output to a remote processing unit for display to a user.

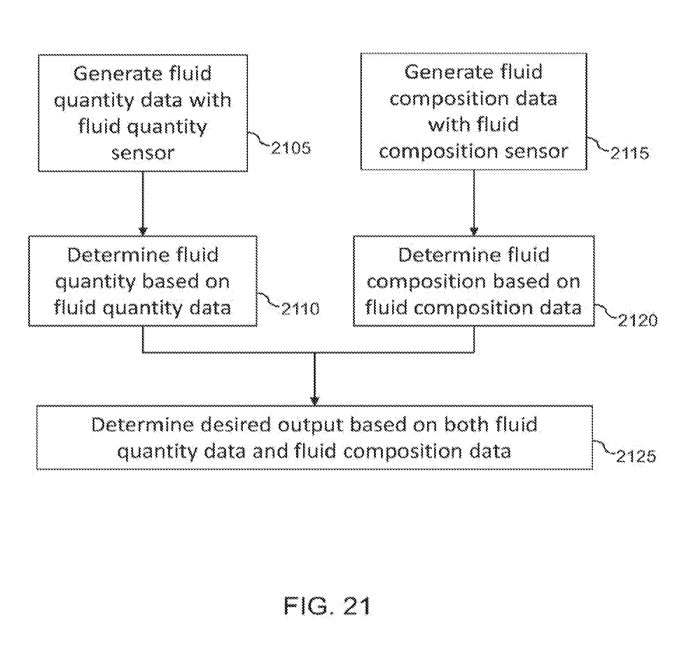

FIG. 21 shows an exemplary method 2100 of determining a desired output value relating to a sample fluid contained in a sensing reservoir in accordance with any embodiment disclosed herein, based on data generated by the sensing reservoir.

In step 2105, fluid quantity data is generated with a fluid quantity sensor. For example, a fluid level sensor comprising a capacitive sensor array may be interrogated to measure the level of fluid inside the reservoir. Optionally, an orientation sensor onboard the reservoir may also be interrogated to determine the orientation of the reservoir during measurement with the capacitive sensor array.

In step 2110, the quantity of fluid contained inside the reservoir may be determined based on the fluid quantity data generated in step 2105. For example, the total volume of fluid present inside the reservoir may be determined based on measured fluid level and orientation of the reservoir.

In step 2115, fluid composition data is generated with a fluid composition sensor. For example, the sensing reservoir may comprise an optical sensing unit configured to generate an absorbance spectrum of the sample, or an electrical conductance sensor configured to measure an electrical conductivity of the sample.

In step 2120, composition of the sample may be determined based on the fluid composition data generated in step 2115. For example, based on measured absorbance spectra of the sample and known spectral signatures of specific components of interest present in the sample fluid, the relative amount of the components present in the sample fluid may be derived. Electric conductivity measurements may be used to derive the salinity of the sample fluid, based on known electrical conductance properties of the sample fluid having known levels of salinity.

In step 2125, the desired output may be determined based on both fluid quantity and composition data generated in steps 2110 and 2120. For example, the total caloric content of a sample of breast milk may be determined based on the percentage of fats, proteins, and lactose present in the sample (as derived from the absorption spectrum of the sample), the known caloric content of each component, and the total volume of the sample present inside the reservoir.

One or more steps of the method 2100 may be performed with circuitry as described herein, for example, a processing unit of a sensing reservoir as described herein, or a remote processing unit in communication with the local processing unit onboard the sensing reservoir. The circuitry may be programmed to provide one or more steps of the method 2100, and the program may comprise programmed instructions stored on a computer readable memory. A person of ordinary skill in the art will recognize many variations of the method 2100, based on the teachings described herein. For example, the steps may be completed in a different order. One or more steps may be added or omitted. Some of the steps may comprise sub-steps. Many of the steps may be repeated as often as necessary or desired. Different steps may be performed by different processing units (local or remote).

In any of the embodiments disclosed herein, the sensing reservoirs described herein can be configured to communicate with another entity, such as one or more computing devices and/or servers. Exemplary computing devices include personal computers, laptops, tablets, and mobile devices (e.g., smartphones, cellular phones). The servers can be implemented across physical hardware, virtualized computing resources (e.g., virtual machines), or any suitable combination thereof. For example, the servers may comprise distributed computing servers (also known as cloud servers) utilizing any suitable combination of public and/or private distributed computing resources. The computing devices and/or servers may be in close proximity to the sensing reservoir and the pumping device (short range communication), or may be situated remotely from the sensing reservoir and the pumping device (long range communication). Any description herein relating to communication between a computing device and a sensing reservoir can also be applied to communication between a server and a sensing reservoir, and vice-versa.

The sensing reservoir can communicate with another computing device via a communication module, as described herein. The communication module can utilize any communication method suitable for transmitting data, such as a wired communication (e.g., wires, cables such as USB cables, fiber optics) and/or wireless communication (Bluetooth®, WiFi, near field communication). In many embodiments, data can be transmitted over one or more networks, such as local area networks (LANs), wide area networks (WANs), telecommunications networks, the Internet, or suitable combinations thereof.

The computing device may be associated with data stores for storage of the measurement data and/or analysis results. Applications of the computing device can also collect and aggregate the measurement data and/or analysis results and display them in a suitable format to a user (e.g., charts, tables, graphs, images, etc.). Preferably, the application includes additional features that allow the user to overlay information such as lifestyle choices, diet, and strategies for increasing milk production, in order to facilitate the comparison of such information with milk production statistics. The analysis and display functionalities described herein may be performed by a single entity, or by any suitable combination of entities. For example, in many embodiments, data analysis can be carried out by a server, and the analysis results may be transmitted to another computing device for display to the user.

Other types of data can also be transmitted between the sensing reservoir and other computing devices. For example, in any embodiment, firmware updates for one or more components of the sensing reservoir can be transmitted to the reservoir from the computing device.

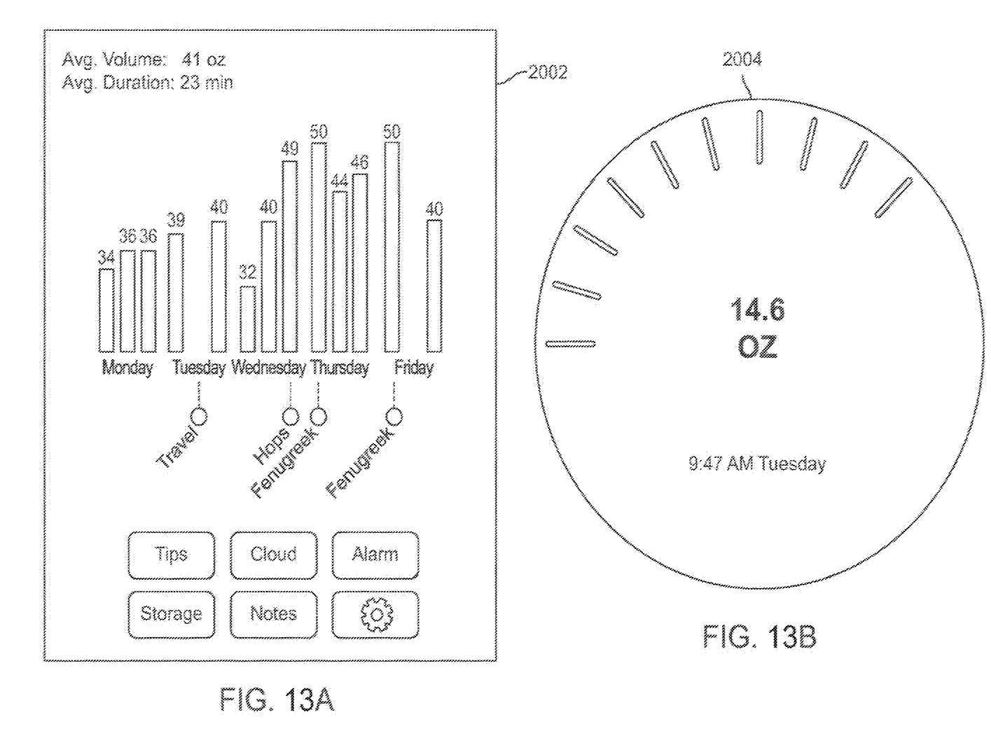

FIGS. 12A-12C illustrate exemplary computing device displays 1904. For example, FIG. 12A illustrates an exemplary display on a mobile phone 1902 and graphically illustrates milk production, the time of the last pumping session, a graphic of goal attainment, and a graphic illustrating the fluid consumption of the user. Additionally, the display 1904 may also provide user encouragement or user feedback based on the amount of milk production. FIG. 12B is an enlarged view of the display 1904 in FIG. 12A. FIG. 12C illustrates additional information that the display 1904 may show when a touch screen is actuated (e.g. by swiping or touching the screen). For example, the volume of the milk expressed is indicated after the “Last Pumping Session” section of the display is selected. Some or all items may be expanded, as also indicated in FIG. 12C. Additional information, or in some situations, less information may be displayed as desired.

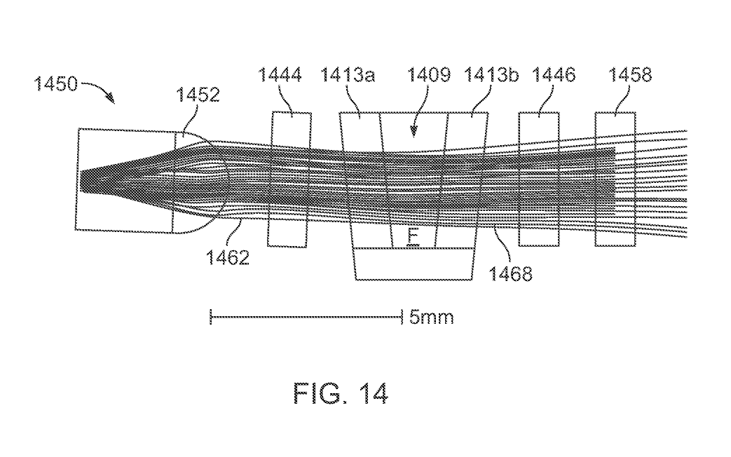

FIGS. 13A-13B illustrate other exemplary displays which may be used in a milk expression system, including any of those disclosed herein. For example, FIG. 13A is an exemplary display 2002 on any of the computing devices disclosed herein and operably coupled with any of the pump units described herein. The display may indicate an average volume of milk expressed over any time period, along with an average duration of the expression session during that same time period. Graphics may be used (e.g. bar chart, pie chart, x-y plot, etc.) to show volume expressed during individual sessions over the course of several days, here Monday through Friday, or any other range of days which may include weekend days. The display may allow a user to annotate the display so that missed sessions may be accounted for, for example if a session is omitted due to traveling, the display may show travel during that time period. Other annotations may also be made, such as when certain foods or nutritional supplements are taken, here hops or fenugreek. This allows the user to recall when expressed milk samples were obtained relative to the consumption of the food or nutritional supplements. The display may have other functional buttons such as for obtaining tips, accessing the cloud, setting an alarm, making notes, storing data, or establishing system preferences. FIG. 13B illustrates another exemplary display 2004 of the computing device. The display 2004 is similar to a dashboard style gauge and indicates the volume of fluid expressed and collected and the time. Other information may also be displayed.

The various techniques described herein may be partially or fully implemented using code that is storable upon storage media and computer readable media, and executable by one or more processors of a computer system. Storage media and computer readable media for containing code, or portions of code, can include any appropriate media known or used in the art, including storage media and communication media, such as but not limited to volatile and non-volatile, removable and non-removable media implemented in any method or technology for storage and/or transmission of information such as computer readable instructions, data structures, program modules, or other data, including RAM, ROM, EEPROM, flash memory or other memory technology, CD-ROM, digital versatile disk (DVD) or other optical storage, magnetic cassettes, magnetic tape, magnetic disk storage or other magnetic storage devices, solid state drives (SSD) or other solid state storage devices, or any other medium which can be used to store the desired information and which can be accessed by the a system device. Based on the disclosure and teachings provided herein, a person of ordinary skill in the art will appreciate other ways and/or methods to implement the various embodiments.

It shall be understood that different aspects of the invention can be appreciated individually, collectively, or in combination with each other. Suitable elements or features of any of the embodiments described herein can be combined or substituted with elements or features of any other embodiment.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. An apparatus for containing and measuring a fluid, the apparatus comprising: a reservoir configured to contain the fluid; and
an optical sensing unit operably coupled to the reservoir, the optical sensing unit configured to generate measurement data indicative of one or more properties of the fluid,
wherein the optical sensing unit comprises a light source and a detector, the light source configured to emit light towards the reservoir, and the detector configured to detect an intensity of the light emanating from the reservoir.

2. The apparatus of claim 1, wherein the light source and the detector are arranged such that the light from the light source travels through the fluid over a path length that is less than 10 mm.

3. The apparatus of claim 2, wherein the path length is less than 5 mm.

4. The apparatus of claim 3, wherein the path length is in a range from about 1 mm to about 5 mm.

5. The apparatus of claim 1, wherein the light is configured to enter the reservoir at a first location of the reservoir and exit the reservoir at a second location of the reservoir positioned across the first location.

6. The apparatus of claim 5, wherein the light source and the detector are arranged such that the first location is on a side wall of the reservoir, and the second location is on a bottom wall of the reservoir, such that the light travels through the fluid across a bottom corner of the reservoir.

7. The apparatus of claim 6, wherein the light from the light source is configured to pass through the first location at an oblique, downward-facing angle towards the second location.

8. The apparatus of claim 7, wherein the reservoir comprises an input light guiding structure configured to direct the light from the light source at the oblique, downward-facing angle.

9. The apparatus of claim 7, wherein the reservoir comprises an output light guiding structure configured to direct the light exiting through the second location towards the detector.

10. The apparatus of claim 5, wherein the reservoir is shaped to provide a channel disposed along a bottom wall of the reservoir and protruding below the bottom wall, the channel comprising a width extending between the first location and the second location.

11. The apparatus of claim 10, wherein the channel is formed by one or more vertical channel walls coupled to a bottom channel wall.

12. The apparatus of claim 10, wherein the channel comprises a material configured to absorb at least a portion of light incident on the channel.

13. The apparatus of claim 10, wherein the light source is configured to emit light directly towards the first location, and wherein the detector is configured to directly receive light emanating from the second location.

14. The apparatus of claim 10, wherein the optical sensing unit further comprises a first lens disposed between the light source and the first location and a second lens disposed between the second location and the detector, wherein the first lens is configured to direct light from the light source towards the first location, and the second lens is configured to direct light from the second location towards the detector.

15. The apparatus of claim 10, wherein the optical sensing unit further comprises a first light guide disposed between the light source and the first location and a second light guide disposed between the second location and the detector, the first light guide configured to direct light from the light source towards the first location, and the second light guide configured to direct light from the second location towards the detector.

16. The apparatus of claim 14, wherein the first light guide is configured to output light in a direction that is substantially parallel to the width of the channel.

17. The apparatus of claim 5, wherein the sensing reservoir further comprises one or more fluid level sensors configured to generate measurement data indicative of a level of fluid contained in the reservoir, wherein the sensing reservoir further comprises a processing unit operatively coupled to the one or more fluid level sensors and the optical sensing unit, and wherein the processing unit is configured with instructions to initiate measurement with the optical sensing unit only if the level of fluid contained in the reservoir exceeds a pre-determined threshold level.

18. The apparatus of claim 1, wherein the optical sensing unit is configured to measure light scattered by the fluid contained in the reservoir.

19. The apparatus of claim 18, wherein the sensing reservoir further comprises one or more fluid level sensors configured to generate measurement data indicative of a level of fluid contained in the reservoir, wherein the sensing reservoir further comprises a processing unit operatively coupled to the one or more fluid level sensors and the optical sensing unit, and wherein the processing unit is configured with instructions to adjust a signal measured by the detector in response to the level of fluid contained in the reservoir.

20. The apparatus of claim 1, further comprising a processing unit operably coupled with the optical sensing unit, wherein the processing unit is configured to one or more of store, process, or transmit to a remote processing unit the measurement data generated by the optical sensing unit.

21. The apparatus of claim 20, further comprising one or more fluid sensors configured to generate measurement data indicative of a level of fluid contained in the reservoir, the one or more fluid sensors operably coupled with the processing unit, wherein the processing unit is configured with instructions to control measurement with the optical sensing unit in response to the level of fluid contained in the reservoir.

22. The apparatus of claim 20, wherein the processing unit is configured with instructions to calibrate a signal measured by the detector to generate the measurement data that is relative with respect to a calibrated value.

23. The apparatus of claim 20, wherein the processing unit is configured with instructions to determine one or more of a composition of the fluid, a nutritional value of the fluid, or a quality of the fluid, based on the measurement data generated by the optical sensing unit.

24. The apparatus of claim 1, wherein the apparatus comprises a plurality of detectors, each of the plurality of detectors configured to receive light having a unique wavelength range, thereby enabling measurement of light absorption by the fluid at a plurality of different wavelengths.

25. The apparatus of claim 24, wherein the optical sensing unit further comprises a plurality of narrow bandpass filters disposed between the reservoir and the plurality of detectors.

26. The apparatus of claim 24, wherein the optical sensing unit comprises a plurality oflight sources, each of the plurality oflight sources configured to emit light having a unique wavelength range, wherein each of the plurality of light sources is aligned with each of the plurality of detectors such that each pair of light source and detector forms a measurement channel for light absorption by the fluid at a unique wavelength range.

27. The apparatus of claim 26, wherein the optical sensing unit further comprises a plurality of narrow bandpass filters disposed between the plurality of light sources and the reservoir.

28. The apparatus of claim 24, further comprising a processing unit operably coupled with the optical sensing unit, wherein the processing unit is configured with instructions to generate a discrete absorption spectrum or a continuous absorption spectrum of the fluid based on the measurement data.

29. The apparatus of claim 1, further comprising a pulsed driver circuit operably coupled with the light source and configured to pulse the light source during measurement with the optical sensing unit, thereby generating measurement data comprising light and dark current measurements.

30. The apparatus of claim 29, further comprising a processing unit operably coupled with the optical sensing unit, the processing unit configured with instructions to adjust a signal measured by the detector in response to dark current measurements.

31.-64. (canceled)
